SEQUENCE LISTING (23 sequences)

SEQ ID NO: 1 - DNA, 1683 bp, Clostridium sp. 14774
cagaattaagaaacacacgtcaaagaaggatccggaaacggatagaagagaaaaagaagagctaaacaaacgaaagattgtaaagataaagagacaatggagagtttgatcctggctcaggatgaacgctggcggcgtgcctaatacatgcaagtcgaacgcgggcagcaatgcccgagtggcgaacgggtgagtaatacataagtaacctgccctttacagggggataactattggaaacgatagctaagaccgcataggtaaagataccgcatggtaagtttattaaaagtgccaaggcactggtagaggatggacttatggcgcattagctagttggtgaggtaacggctcaccaaggcgacgatgcgtagccgacctgagagggtgaccggccacactgggactgagacacggcccagactcctacgggaggcagcagtagggaattttcggcaatggggggaaccctgaccgagcaacgccgcgtgaaggaggaaggtcttcggactgtaaacttctgttataaaggaagaaaggcggatacagggaatggtatccgagtgacggtactttatgaggaagccacggctaactacgtgccagcagccgcggtaatacgtaggtggcaagcgttatccggaattattgggcgtaaagagggagcaggcggcagcaagggtctgtggtgaaagactgaagcttaacttcagtaagccatagaaaccgggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttgggagtcaaatctcagtgctgcagttaacgcagtaagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagatatatgggatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaaggcggggatgacgtcaaatcatcatgccccttatgacctgggctacacacgtgctacaatggatggagcagagggaagcgaagccgcgaggtggagcgaaacccagaaaaccattctcagttcggattgtagtctgcaactcgactacatgaagttggaatcgctagtaatcgcgaatcagcatgtcgcggtgaatacgttctcgggccttgtacacaccgcccgtcacaccatgagagttgataacacccgaagccggtggcctaaccgcaaggagggagctgtctaaggtgggattgatgattggggtgaagtcgtaacaaggtatccctacgggaacgtggggatggatcacctcctttctaaggagaaagaagagaagtgaagtttcattaatactgtttagttttgagtgatgctcaggagg

SEQ ID NO: 2 - DNA, 1685 bp, Lachnospiraceae 7 1 58FAA
ttaagcttcgataagatgatttgaggaacggagacgttctttagataccattttattgagagtttgatcctggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctcctttctaaggagacttcaacggaagtagttttctgttgtaacgtcctaaggtcagctttctggagcgaatggctttcacgttgtttaatttagagggtacactctc

SEQ ID NO: 3 - DNA, 1757 bp, Hungatella hathewayi
attcagaacgaatacaattattgtatacgaaccaaacaacaagtaaaacgggaaataaattagctagttagttgattttgaccccggatcaaacgaatcatttaacatgagagttcgatcctggctcaggatgaacgctggcggcgtgcttaacacatgcaagtcgagcgaagcggtttcaatgaagttttcggatggaattgaaattgacttagcggcggacgggtgagtaacgcgtgggtaacctgccttacactgggggataacagttagaaatgactgctaataccgcataagcgcacagggccgcatggtctggtgtgaaaaactccggtggtgtaagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggttaagcaagtctgaagtgaaagcccggggctcaaccccggtactgctttggaaactgtttgacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcgggggacaacgtccttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccattgaaaatcatttaaccgtgatccctcttcggagcaatggagacaggtggtgcatggttgtcgtcagctcgtgtcgtgagatgttgggttaagtcccgcaacgagcgcaacccttatccttagtagccagcacatgatggtgggcactctggggagactgccagggataacctggaggaaggtggggatgacgtcaaatcatcatgccccttatgatttgggctacacacgtgctacaatggcgtaaacaaagggaagcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgtaaggagggagctgccgaaggcgggactgataactggggtgaagtcgtaacaaggtagccgtatcggaaggtgcggctggatcacctcctttctaaggaagaagaagtaagggttttatatactgttgagtcttgtgtttcaaaggaaggcgaaagccgaagaaaaatagaataaaagaatagaaacatcagagacaaagagattctggtgccga

SEQ ID NO: 4 - DNA, 2632 bp, Blautia producta
cgaagtctggtgtggaatattcagacaccccaaattttttcatacactgcgatccgggaagcatatgaggaagcgcggcagcagagcatggaggatattacagatgatgccatggtcatggagcgctttggaaacttgaaaattaagttggtggaaggttcctatgagaacattaaaatcactacgccggaggacattcttgtcgctgagaaaattttggaaaaaaattagagaaatctattgacaacttccgacataagtgataagataccacttgtcgctgaccgagtgacaaacatggagagatatcgaagtggtcataacgaggcggtcttgaaaaccgtttgtccgcaagggcgcgtgggttcgaatcccactctctccgttcccgaaagcagatatcgagaacgaatcagagtgacgagttcaagattagttatgatgcttttggatatatagagaagcagttatttgaaagcttactttcaaactgttgttttattcagcctggagaagtacccaagcggctgaaggggctcccctggaaagggagtaggtcgttaatagcggcgcgagggttcaaatccctccttctccgttttctcttaaaaaaagagaaaaaagttgttgacaagcgatcggatatatgataatctattatagctgtcgcaaaaacgacaacgatttatgacattgaaaatgagctttgcttaaaagctttgtgagtaaatgcaaaggtttagcgtagttgaaagaagtttcgacacaacgtaaaaaaacttttgaaaaacaatacaattaaaaataaatttttaattgtcataagttcttgacaagcacgaaacgatatgataaaatatcaaagctgtcaaaaactgacaggaaccttgataactgaacagtgaaacacatgaatcgaaaattcttttacattcattagtaacaaaacgaacggttctaaacgaaccaaaacagtaaaagggatatcttattgagcctagctcaataagccagaattagccaaagttaattctgttcctggacaaacacttgatcagagagtttgatcctggctcaggatgaacgctggcggcgtgcttaacacatgcaagtcgagcgaagcattaagacagatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaatgggggcttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaggcgggaccgataactggggtgaagtcgtaacaaggtagccgtatcggaaggtgcggctggatcacctcctttctaaggaagaagaagtagagaaaagtgtttcactgttgagttaccaaga

SEQ ID NO: 5 - DNA, 1702 bp, Clostridium bolteae
gaaaattcagaacaaaaccaagtaatgaaggttaaagataactagccaagctaagtagtctgaaacctggaatcaacttttaaacatgagagtttgatcctggctcaggatgaacgctggcggcgtgcctaacacatgcaagtcgaacgaagcaattaaaatgaagttttcggatggatttttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctcctttctaaggaagaagaagtaggggtttatgtattattgagtccttgattaaaaggtgcttaaaattttccggtggcgatgcgcttagggg

SEQ ID NO: 6 - DNA, 1494 bp, Clostridiaceae JC118
agagtttgatcctggctcaggatgaacgctggcggcatgcctaatacatgcaagtcgaacgaagtgaagatagcttgctatcggagcttagtggcgaacgggtgagtaacacgtagataacctgcctgtatgaccgggataacagttggaaacgactgctaataccggataggcagagaggaggcatctcttctctgttaaagttgggatacaacgcaaacagatggatctgcggtgcattagctagttggtgaggtaacggcccaccaaggcgatgatgcatagccggcctgagagggcgaacggccacattgggactgagacacggcccaaactcctacgggaggcagcagtagggaattttcggcaatgggggaaaccctgaccgagcaatgccgcgtgagtgaagacggccttcgggttgtaaagctctgttgtaagggaagaacggcatagagagggaatgctctatgagtgacggtaccttaccagaaagccacggctaactacgtgccagcagccgcggtaatacgtaggtggcaagcgttatccggaattattgggcgtaaagggtgcgtaggcggctggataagtctgaggtaaaagcccgtggctcaaccacggtaagccttggaaactgtctggctggagtgcaggagaggacaatggaattccatgtgtagcggtaaaatgcgtagatatatggaggaacaccagtggcgaaggcggttgtctggcctgtaactgacgctgaagcacgaaagcgtggggagcaaataggattagataccctagtagtccacgccgtaaacgatgagaactaagtgttggggaaactcagtgctgcagttaacgcaataagttctccgcctggggagtatgcacgcaagtgtgaaactcaaaggaattgacgggggcccgcacaagcggtggagtatgtggtttaattcgacgcaacgcgaagaaccttaccaggccttgacatggtatcaaaggccctagagatagggagatagttatgatacacacaggtggtgcatggttgtcgtcagctcgtgtcgtgagatgttgggttaagtcccgcaacgagcgcaacccttgtttctagttaccaacagtaagatggggactctagagagactgccggtgacaaaccggaggaaggtggggatgacgtcaaatcatcatgccccttatggcctgggctacacacgtactacaatggcgtctacaaagagcagcgagcaggtgactgtaagcgaatctcataaaggacgtctcagttcggattgaagtctgcaactcgacttcatgaagtcggaatcgctagtaatcgcggatcagcatgccgcggtgaatacgttctcgggccttgtacacaccgcccgtcaaaccatgggagttgataatacccgaagccggtggcctaaccgaaaggagggagccgtcgaaggtaggatcgatgactggggttaagtcgtaacaaggtatccc

SEQ ID NO: 7 - DNA, 1703 bp, Anaerostipes unclassified
aaaaaagatacacgaacgttctttataaaaggacacagtaaaacaaggataaaaaagctagcgctttttagatccaagattgaacatgagagtttgatcctggctcaggatgaacgctggcggcgtgcttaacacatgcaagtcgaacgaagcattttggaaggaagttttcggatggaattccttaatgactgagtggcggacgggtgagtaacgcgtggggaacctgccctatacagggggataacagctggaaacggctgctaataccgcataagcgcacagaatcgcatgattcggtgtgaaaagctccggcagtataggatggtcccgcgtctgattagctggttggcggggtaacggcccaccaaggcgacgatcagtagccggcttgagagagtggacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaaaagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaacgcagtaagtattccacctggggagtacgttcgcaagaatgaaactcaaaggaattgacggggacccgcacaagcggtggagcatgtggtttaattcgaagcaacgcgaagaaccttacctggtcttgacatctaactgaccggttcgtaatgggacctttccttcgggacagttaagacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatggccagggctacacacgtgctacaatggcgtaaacaaagggaagcgaagtcgtgaggcgaagcaaatcccagaaataacgtctcagttcggattgtagtctgcaactcgactacatgaagctggaatcgctagtaatcgtgaatcagaatgtcacggtgaatacgttcccgggtcttgtacacaccgcccgtcacaccatgggagtcagtaacgcccgaagtcagtgacccaaccttataggagggagctgccgaaggtgggaccgataactggggtgaagtcgtaacaaggtagccgtatcggaaggtgcggctggatcacctcctttctaaggaagaagaagtaggggttgtcttattgtttagttatcaatgataacaaaaagtatcaagcaaggaagttctattgcactaaa

SEQ ID NO: 8 - DNA, 1571 bp, Anaerotruncus colihominis
atcaaagagtttgatcctggctcaggacgaacgctggcggcgcgcctaacacatgcaagtcgaacggagcttacgttttgaagttttcggatggatgaatgtaagcttagtggcggacgggtgagtaacacgtgagcaacctgcctttcagagggggataacagccggaaacggctgctaataccgcatgatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcatagcctagagataggtgaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactaaaacagagggcggcgacaccgcgaggtgaagcgaatcccgaaaaagtgtctcagttcagattgcaggctgcaacccgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggggggcgctgtcgaaggtgggattgatgactggggtgaagtcgtaacaaggtagccgtatcggaaggtgcggctggatcacctcctttctaaggagcgaagccggcggagagccggcggactctggtcagacaggg

SEQ ID NO: 9 - DNA, 1700 bp, Ruminococcus sp. ID8
aacaggaactttaagttccacaaaccagacaacagtaacgggatagattagcgagagttgatcttgaccagaacaagttcttattacgagagtttgatcctggctcaggatgaacgctggcggcgtgcctaacacatgcaagtcgagcgaagcgctgttttcagaatcttcggaggaagaggacagtgactgagcggcggacgggtgagtaacgcgtgggcaacctgcctcatacagggggataacagttagaaatgactgctaataccgcataagcgcacaggaccgcatggtgtagtgtgaaaaactccggtggtatgagatggacccgcgtctgattaggtagttggtggggtaaaggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaagatgacggtacctgagtaagaagcaccggctaaatacgtgccagcagccgcggtaatacgtatggtgcaagcgttatccggatttactgggtgtaaagggagcgtagacggataggcaagtctggagtgaaaacccagggctcaaccctgggactgctttggaaactgcagatctggagtgccggagaggtaagcggaattcctagtgtagcggtgaaatgcgtagatattaggaggaacaccagtggcgaaggcggcttactggacggtgactgacgttgaggctcgaaagcgtggggagcaaacaggattagataccctggtagtccacgccgtaaacgatgactactaggtgtcggtgtgcaaagcacatcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctggtcttgacatccggatgacgggcgagtaatgtcgccgtcccttcggggcatccgagacaggtggtgcatggttgtcgtcagctcgtgtcgtgagatgttgggttaagtcccgcaacgagcgcaacccttatcttcagtagccagcatataaggtgggcactctggagagactgccagggagaacctggaggaaggtggggatgacgtcaaatcatcatgccccttatggccagggctacacacgtgctacaatggcgtaaacaaagggaagcgagagggtgacctggagcgaatcccaaaaataacgtctcagttcggattgtagtctgcaactcgactacatgaagctggaatcgctagtaatcgcggatcagcatgccgcggtgaatacgttcccgggtcttgtacacaccgcccgtcacaccatgggagtcagtaacgcccgaagccagtgacccaaccttagaggagggagctgtcgaaggcgggacggataactggggtgaagtcgtaacaaggtagccgtatcggaaggtgcggctggatcacctcctttctaagggaagaagtaagggacgttgtctattgttgagcgatcgaagaggggagacccgaagaggagctcagaagcagttggtggtga

SEQ ID NO: 10 - DNA, 1751 bp, Clostridium asparagiforme
gaaccttgaaaatcaaagattgaacagtatgtaaaaccctgaaaattctaaaaaataaaggtctggtttagacctttggatttgagaaaattcagaacaaaaccaagtaatgaaggttaaagataactagccaagctaagtagtctaagaccaagaatcaacttttaatttgagagtttgatcctggctcaggatgaacgctggcggcgtgcctaacacatgcaagtcgaacgaagcattttagatgaagttttcggatggattctgagatgactgagtggcggacgggtgagtaacacgtggataacctgcctcacactgggggacaacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcatggcaagtctgaagtgaaaacccagggctcaaccctgggactgctttggaaactgtcaagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgagtgctaggtgttggggggcaaagcccttcggtgccgtcgcaaacgcaataagcactccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgcctttccttcgggacaagagagacaggtggtgcatggttgtcgtcagctcgtgtcgtgagatgttgggttaagtcccgcaacgagcgcaacccttatccttagtagccagcattaagatgggcactctagggagactgccagggacaacctggaggaaggtggggatgacgtcaaatcatcatgccccttatgatttgggctacacacgtgctacaatggcgtaaacaaagggaagcgaccctgcgaaggtgagcaaatctcaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgtaacaaggtagccgtatcggaaggtgcggctggatcacctcctttctaaggaagaagaagtaagggttttatatactgttgaatcttggatttgccaaggt

SEQ ID NO: 11 - DNA, 2795 bp, Clostridium 7 3 54FAA
ccggatcaatatgtaggtttaaaaacaaaggaaatcgcagatatagtgcacgatcggattcaaaacaagattgatgaaaatatataaaaatatctggaaaaacaggcttgacaacccaattcttatttagtataattgaccatgcgcttgacatgagcatgtccaagccgcatagttcaacttaatagttttaagtaataactgcggagaaatactcaagaggccgaagaggcgcccctgctaagggtgtaggtcgggcaaccggcgcgagggttcaaatccctctttctccgttttcttttttgaaaaaaaacctattgaatatattgtttgaaaaacttcaaaaaaggtgttgacaccgaatgaaagttatgatatgatattcaagctgctgaaaacgagcggcggtttcaaaaaaagaaattaaaaaaataaaaaaagttgttgacaaacaaaaacaattatggtaagatataaaagttgctgctgagaacaaacgacagcgttcaacaacaaagaaccttgaaaactgaacagtatgtaaaaccttgaaaattcttaaagaacaaaggcttttggttaagcctaaaagttcgagagaacattcagaacaaaaaaaccaaacaacagtaatacggtttaaaaattagccaagcgaatttttgaacggaatcaaaacttaaacatgagagtttgatcctagctcaggatgaacgctggcggcgtgcctaacacatgcaagtcgaacgaagcaatttaacggaagttttcggatggaagttgaattgactgagtggcggacgggtgagtaacgcgtgggtaacctgccttgtactgggggacaacagttagaaatgactgctaataccgcataagcgcacagtatcgcatgatacagtgtgaaaaactccggtggtacaagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtggcgaaggcgacttactggacgataactgacgttgaggctcgaaagcgtggggagcaaacaggattagataccctggtagtccacgccgtaaacgatgaatactaggtgttggggagcaaagctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatcgatccgacgggggagtaacgtccccttcccttcggggcggagaagacaggtggtgcatggttgtcgtcagctcgtgtcgtgagatgttgggttaagtcccgcaacgagcgcaacccttattctaagtagccagcggttcggccgggaactcttgggagactgccagggataacctggaggaaggtggggatgacgtcaaatcatcatgccccttatgatctgggctacacacgtgctacaatggcgtaaacaaagagaagcaagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggcgggaccgataactggggtgaagtcgtaacaaggtagccgtatcggaaggtgcggctggatcacctcctttctaaggaagaagaagtagaggttttatatactgttgagttttcaagctcagacacttccggtgtcgatgcgctcaggggtcacacccgttcccatcccgaacacgatggttaagacctgagcggccgatgatactatattggagacgatatgggaaagcaggtggatgccggatttttctttaaaaaactcactcgtaagagtgttttatatagaacggacacatctgaatggatgaattcgtagtttctttagatgaaaaagacatctgtgacgacgatgtgttaacccactccagcagggaagtgctgttccatataactggtttttaccagtgattagtgaatttgagtttatcgagtttgctaatgactgataaagttcagtcagatatgtaccttgaaaactgcatattgaaataatatctagatagagtttcgaaactcttccgagtggcttgccacaagggagaaaagagagacaaaatcaagacatccgaggtgttacacgaaagtgtaaccaaaacaaaccaaaaattttattgtaaacctaaaccaaagattcaacgctatgaatcttagact

SEQ ID NO: 12 - DNA, 1704 bp, Coprobacillus unclassified
attgaaaactaaacagaattaagaaacacacgtcaatatccagaaggataaaaaagaaaaaagagctaaacaaacgaagagttgtttaaataaacaatggagagtttgatcctggctcaggatgaacgctggcggcgtgcctaatacatgcaagtcgaacgcgagcacttgtgctcgagtggcgaacgggtgagtaatacataagtaacctgccctagacagggggataactattggaaacgatagctaagaccgcataggtacggacactgcatggtgaccgtattaaaagtgcctcaaagcactggtagaggatggacttatggcgcattagctggttggcggggtaacggcccaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaaggttttcggattgtaaacttctgttataaaggaagaacggcggctacaggaaatggtagccgagtgacggtactttattagaaagccacggctaactacgtgccagcagccgcggtaatacgtaggtggcaagcgttatccggaattattgggcgtaaagagggagcaggcggcagcaagggtctgtggtgaaagcctgaagcttaacttcagtaagccatagaaaccaggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttggatgtcaaagttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagctatatgagatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaaggcggggatgacgtcaaatcatcatgccccttatgacctgggctacacacgtgctacaatggatggtgcagagggaagcgaagccgcgaggtgaagcaaaacccataaaaccattctcagttcggattgtagtctgcaactcgactacatgaagttggaatcgctagtaatcgcgaatcagcatgtcgcggtgaatacgttctcgggccttgtacacaccgcccgtcacaccacgagagttgataacacccgaagccggtggcctaaccgcaaggaaggagctgtctaaggtgggattgatgattggggtgaagtcgtaacaaggtatccctacgggaacgtggggatggatcacctcctttctaaggagaaacgcatgatcgaagatgatgcacaagagaagtgatgtttcgaagatactgtttagttttgagtgatactcaggt

SEQ ID NO: 13 - DNA, 1645 bp, Eubacterium fissicatena
actgagattaatctcggtggtaagaacagtaaaaagggatagaattagctagtagttaattctgacccggatacaaacacttttaacgagagtttgatcctggctcaggatgaacgctggcggcgtgcttaacacatgcaagtcgagcgaagcgctttacttagatttcttcggattgaaaagttttgcgactgagcggcggacgggtgagtaacgcgtgggtaacctgcctcatacagggggataacagttagaaatgactgctaataccgcataagaccacagtaccgcatggtacagtgggaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggttatgtaagtctgatgtgaaaacccggggctcaaccccgggactgcattggaaactatgtaactagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgcatactaggtgtcgggtggcaaagccattcggtgccgcagcaaacgcaataagtatgccacctggggagtacgttcgcaagaatgaaactcaaaggaattgacggggacccgcacaagcggtggagcatgtggtttaattcgaagcaacgcgaagaaccttacctgctcttgacatccccctgaccggcgcgtaatggtgcctttccttcgggacaggggagacaggtggtgcatggttgtcgtcagctcgtgtcgtgagatgttgggttaagtcccgcaacgagcgcaacccttatctttagtagccagcggtttggccgggcactctagagagactgccagggataacctggaggaaggtggggatgacgtcaaatcatcatgccccttatgagcagggctacacacgtgctacaatggcgtaaacaaagggaggcgaagccgcgaggtggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagttggtaacgcccgaagtcagtgacccaaccgcaaggagggagctgccgaaggtgggaccgataactggggtgaagtcgtaacaaggtagccgtatcggaaggtgcggctggatcacctcctttctaaggaagaaagaagtaaagagtatgttgt

SEQ ID NO: 14 - DNA, 1761 bp, Lachnospiraceae bacterium 5_1_57FAA
tcaaccacagaaccttgataattgaacaatagacaacaaccctgaaaatttcttgtaaagagagaatttcagaacagaacgggacatcgagagatgtccgaccttaaaaacagtaataacgggaaagaattagccaagagttgattctgaccgcgatcacaacttttaacgagagtttgatcctggctcaggatgaacgctggcggcgtgcctaacacatgcaagtcgaacgaagcgcttccgctagattttcttcggagatgaaggcggctgcgactgagtggcggacgggtgagtaacgcgtgggcaacctgccttgcactgggggataacagccagaaatggctgctaataccgcataagaccgaagcgccgcatggcgcagcggccaaagccccggcggtgcaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgagatgttgggttaagtcccgcaacgagcgcaacccctatcttcagtagccagcattccggatgggcactctggagagactgccagggacaacctggaggaaggtggggatgacgtcaaatcatcatgccccttatgaccagggctacacacgtgctacaatggcgtaaacaaagggaggcgaacccgcgagggtgggcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagccggtgacccaacccgtatgggagggagccgtcgaaggtgggaccgataactggggtgaagtcgtaacaaggtagccgtatcggaaggtgcggctggatcacctcctttctaaggaagagaagaagtaagggctgctgtctattgttcagttatcgaggaatgctggaa

SEQ ID NO: 15 - DNA, 1608 bp, Clostridium_sp_KLE_1755
tagagttaatcttaactgggttaaacacttaaacaagagagtttgatcctggctcaggatgaacgctggcggcgtgcctaacacatgcaagtcgaacggagttatgcagaggaagttttcggatggaatcggcgtaacttagtggcggacgggtgagtaacgcgtgggaaacctgccctgtaccgggggataacacttagaaataggtgctaataccgcataagcgcacagcttcacatgaggcagtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatgacaagccagatgtgaaaacccagggctcaaccctgggactgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatcccaatgacgtgtccgtaacggggcattctcttcggagcattggagacaggtggtgcatggttgtcgtcagctcgtgtcgtgagatgttgggttaagtcccgcaacgagcgcaacccttatccttagtagccagcaggtaaagctgggcactctagggagactgccggggataacccggaggaaggcggggatgacgtcaaatcatcatgccccttatgatttgggctacacacgtgctacaatggcgtaaacaaagggaagcgagacagtgatgttgagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagcctgtgacctaaccgtaagggaggagcagtcgaaggcaggtctaataactggggtgaagtcgtaacaaggtagccgtatcggaaggtgcggctggatcacctcctttctaaggcgaagaagtaagggttgtattactgtttagatgttaaggg

SEQ ID NO: 16 - DNA, 1555 bp, Clostridiales 1_7_47FAA
aatttgagagtttgatcctggctcaggatgaacgctggcggcgtgcctaacacatgcaagtcgaacgaagcatcttataggaagttttcggatggaatatgggatgactgagtggcggacgggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgatgcaagtctgaagtgaaagcccggggctcaaccccgggactgctttggaaactgtgtggctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccctcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggacagtaacgtgtcccttccttcgggacaggggagacaggtggtgcatggttgtcgtcagctcgtgtcgtgagatgttgggttaagtcccgcaacgagcgcaacccttatccttagtagccagcacctcgggtgggcactctagggagactgccagggataacctggaggaaggtggggatgacgtcaaatcatcatgccccttatgatttgggctacacacgtgctacaatggcgtaaacaaagggaagcggccctgcgaaggtgagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgtaacaaggtagccgtatcggaaggtgcggctggatcacctcctttctaaggaagaagaagtaggggtttt

SEQ ID NO: 17 - DNA, 2231 bp, Lachnospiraceae 3_1_57FAA
gaattgacaaacagttacgccttatgtataataacatttgcgctaccgaatgggacgcgcacttcatattggtagaggatagttcagttttgtggagaaatactcaagaggctgaagaggcgcccctgctaagggtgtaggtcgtttatagcggcgcgagggttcaaatccctctttctccgttccctttaccgatataaaggggagcttgattatagcttcgcgggaaaagaatcttaagagattgagatttcttttttttcgcacttttgccggatgaaaagaacgagtaaaacttatggaaaatataggttaaaaaagttgttgacatttgacaaaatatggtgctataatgaaagtccaccgcgaatggcgatggaacagagatttaaaagaaataaaaaaagttaaaaaagtgcttgactggcagaacaacatgtgataagatatcaactgttgcggctgagaaagcagcacacagaaccttgacaaataaacagtaatgcaaccctgaaaattctaaaaaagaaaattcagagaacaagttttaaagcttcgagagaagacctttaagacacccaaaaacagtaagaaatgtttggaaacaaacaaacggttaaacattagctagagttaatcttaaccggattaaacacttaaacaagagagtttgatcctggctcaggatgaacgctggcggcgtgcctaacacatgcaagtcgaacgaagttagacagaggaagttttcggatggaatcggtataacttagtggcggacgggtgagtaacgcgtgggaaacctgccctgtaccgggggataacacttagaaataggtgctaataccgcataagcgcacggaaccgcatggttctgtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatcccaatgacgcacctgtaaagaggtgttcccttcggggcattggagacaggtggtgcatggttgtcgtcagctcgtgtcgtgagatgttgggttaagtcccgcaacgagcgcaacccttattcttagtagccagcaggtaaagctgggcactctaaggagactgccggggataacccggaggaaggcggggatgacgtcaaatcatcatgccccttatgatttgggctacacacgtgctacaatggcgtaaacaaagggaagcgagacagtgatgtggagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagtctgtgacctaaccgaaagggaggagcagccgaaggcaggtctgataactggggtgaagtcgtaacaaggtagccgtatcggaaggtgcggctggatcacctcctttctaaggcgaagaagtaggggttgtattactgtttagatgtt

SEQ ID NO: 18 - DNA, 20 bp, Artificial sequence
  Synthetic
aaatgacggtacctgactaa

SEQ ID NO: 19 - DNA, 22 bp, Artificial sequence
  Synthetic
ctttgagtttcattcttgcgaa

SEQ ID NO: 20 - DNA, 16 bp, Artificial sequence
  Synthetic
gcacaagcagtggagt

SEQ ID NO: 21 - DNA, 18 bp, Artificial sequence
  Synthetic
cttcctccgttttgtcaa

SEQ ID NO: 22 - DNA, 20 bp, Artificial sequence
  Synthetic
agagtttgatcmtggctcag

SEQ ID NO: 23 - DNA, 16 bp, Artificial sequence
  Synthetic
attaccgcggckgctg

(19) United States
(12) Patent Application Publication
(10) Pub. No.: US 2025/0270498 A1
(43) Pub. Date: Aug. 28, 2025

(54) OPTIMIZED CULTURE MEDIA FOR CLOSTRIDIA BACTERIA

(71) Applicant: Janssen Biotech, Inc., Horsham, PA (US)

(72) Inventors: John Bodek, Collegeville, PA (US); Kristina Demjanick, Coatesville, PA (US); Laura Grussendorf, Springfield, PA (US); John Jerbasi, Gloucester City, NJ (US); Claus Lang, Hünibach (CH); Dominik Lüthy, Bern (CH); Peter Hebbeln, Mittelhäusern (CH); Shant Shahinian, Upper Darby, PA (US); Stuti Mehta, Lansdale, PA (US); Susan Lenk, Exton, PA (US)

(73) Assignee: Janssen Biotech, Inc., Horsham, PA (US)

(21) Appl. No.: 17/633,993

(22) PCT Filed: Jul. 21, 2020
(86) PCT No.: PCT/IB2020/056846

§ 371 Date: Feb. 9, 2022

Related U.S. Application Data
(60) Provisional application No. 62/887,790, filed on Aug. 16, 2019.

(51) Int. Cl. C12N 1/20; C12R 1/145

(52) U.S. Cl. CPC C12N 1/20; C12R 2001/145

(57) ABSTRACT

The present invention relates to media compositions for culturing bacteria. In particular, the present invention relates to media for culture and/or fermentation of Clostridia bacteria.

### CROSS REFERENCE TO RELATED APPLICATIONS

This application claims the benefit of International Application Number PCT/IB2020/056846, filed 21 Jul. 2020, which claims the benefit of U.S. Provisional Application Ser. No. 62/887,790, filed 16 Aug. 2019. The disclosure of the aforementioned application is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name JBI6144USPCT1Sequencelisting_Corrected.txt, creation date of 25 Aug., 2022 and having a size of 44 KB. The sequence listing submitted via EFS-Web is part of the specification and is herein incorporated by reference in its entirety.

### FIELD OF THE INVENTION

The disclosure provided herein relates to bacterial culture media, useful for growing bacteria belonging to Clostridium genus, and methods of producing and using the described media.

### BACKGROUND OF THE INVENTION

Recent studies have shown that bacteria of the genus Clostridium control differentiation of the host's immune cells in the mucosal immune system in the mammal gut (WO2013080561, WO2011151941). Thus, it may be beneficial to introduce specific bacteria of the genus Clostridium into an organism, in order to modulate the organism's immune system.

In order to develop drugs, dietary supplements, probiotics, or foods, comprising these bacteria, with beneficial immune functions for human use, it is necessary to develop methods to cultivate them so that they can be produced by traditional industrial fermentation processes and subsequently incorporated in pharmaceutical or food formulations. The production of Clostridium bacteria being an expensive process, there is continuous research to improve the growth media used to produce these bacteria with high yield. Additional difficulty arises when several strains of Clostridium bacteria are used in a combination. Growing each of several Clostridium strains in a different medium can be cost-ineffective and time-consuming. It is therefore desirable to develop novel media, optimized for culturing several strains of Clostridium bacteria with high yield.

### SUMMARY OF THE INVENTION

The present invention solves the above identified problems by providing optimized media compositions and processes for culturing bacterial cells.

In one embodiment, the medium comprises: (a) 5-15 g/kg of Proteose Peptone, (b) 5-15 g/kg of Phytone Peptone, (c) 15-25 g/kg of Yeast extract, (d) 9-14 g/kg of M9 salts, (e) 5-15 g/kg of glucose, wherein the medium is for culturing a bacterial strain belonging to Clostridium genus.

In another embodiment, the medium comprises: (a) 10 g/kg of Proteose Peptone, (b) 10 g/kg of Phytone Peptone, (c) 20 g/kg of Yeast extract, (d) 11.4 g/kg of M9 salts, (e) 10 g/kg of glucose.

In another embodiment, the medium is for culturing a bacterial strain belonging to Clostridium genus, wherein the bacterial strain belonging to Clostridium genus is a bacterial strain belonging to Clostridium clusters IV or XIVa.

In another embodiment, the medium is for culturing a bacterial strain belonging to Clostridium genus, wherein the bacterial strain belonging to Clostridium genus is selected from the group consisting of the strains listed in Table 1.

In another embodiment, the medium is for culturing a bacterial strain belonging to Clostridium genus, wherein the bacterial strain belonging to Clostridium genus comprises a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In another embodiment, the medium is maintained in anaerobic conditions.

In another embodiment, the medium further comprises at least one bacterium comprising 16S rDNA sequences selected from the group: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In one embodiment, the process for culturing bacterial cells comprises: a) providing a bioreactor, b) mixing the cells to be cultured with the medium, c) incubating the resultant mixture.

In another embodiment, the process for culturing bacterial cells is the process wherein the bacterial strain belonging to Clostridium genus is a bacterial strain belonging to Clostridium clusters IV or XIVa.

In another embodiment, the process for culturing bacterial cells is the process, wherein the bacterial strain belonging to Clostridium genus is selected from the group consisting of the strains listed in Table 1.

In another embodiment, the process for culturing bacterial cells is the process, wherein the bacterial strain belonging to Clostridium genus comprises a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In another embodiment, the process for culturing bacterial cells is the process, wherein the medium is maintained in anaerobic conditions.

### DETAILED DESCRIPTION OF THE INVENTION

All publications, including but not limited to patents and patent applications, cited in this specification are herein incorporated by reference as though fully set forth.

The disclosed subject matter may be understood more readily by reference to the following detailed description taken in connection with the accompanying figures, which form a part of this disclosure. It is to be understood that the disclosed subject matter is not limited to those described and/or shown herein, and that the terminology used herein is for the purpose of describing particular embodiments by way of example only and is not intended to be limiting of the claimed subject matter.

Unless specifically stated otherwise, any description as to a possible mechanism or mode of action or reason for improvement is meant to be illustrative only, and the disclosed subject matter are not to be constrained by the correctness or incorrectness of any such suggested mechanism or mode of action or reason for improvement.

When a range of values is expressed, another embodiment includes from the one particular value and/or to the other particular value. Further, reference to values stated in ranges include each and every value within that range. All ranges are inclusive and may be combined. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed subject matter which are, for clarity, described herein in the context of separate embodiments, may also be provided in combination in a single embodiment. Conversely, various features of the disclosed subject matter that are, for brevity, described in the context of a single embodiment, may also be provided separately or in any subcombination.

### Definitions

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.10% or less from the specified value.

“Bacterial growth” is defined as the division of a bacterial cell in two identical daughter cells during a process called binary fission. The duplication of bacterial population occurs at each cell division, undergoing exponential growth. Exponential bacterial growth in a culture can be monitored through well-known methods, such as, direct count of bacterial cells (i.e. microscopy, flow cytometry), biomass quantification (milligrams, grams, kilos or tons), colony count, optical density (measured in spectrophotometer, wavelength of about 600 nm), nutrient consumption, among others.

Bacterial growth can be characterized by four different phases: “lag phase”, “exponential or log phase”, “stationary phase” and “decline or death phase”.

It is of common knowledge that during the “lag phase”, the bacteria are adapting to growth conditions and are not yet able to perform cellular division. It is a period when cells are entering a maturity state. The “exponential or log phase” is a period characterized by duplication of bacterial population. If growth is not restricted, cell duplication continues in a constant rate, so both, the number of cells and growth rate, duplicate in each generation. The exponential growth phase is not sustained indefinitely since the growth medium has nutritional restraints and the metabolites produced by bacterial cells during cellular division are often toxic. Thus, the growth rate tends to decrease and the bacterial growth enters in the “stationary phase”. This phase is characterized by resource depletion in the culture medium. In the “decline or death phase”, bacteria deplete completely the remaining nutrients in the culture medium and die.

“Pre-culture” or “pre-inoculum” is defined as a suspension of microorganisms obtained from a stock culture that will be used for “culture” or “inoculum” production.

The terms “culture”, “cell culture”, or “inoculum” are used interchangeably and define a suspension of microorganisms with a specific concentration to be used for growth and/or fermentation on a larger scale (greater volume of culture medium) than the initial one.

A cell culture can be performed in any container suitable for the culture of cells, such as a petri dish, contact plate, bottle, tube, well, vessel, bag, flask or tank. Typically the container is sterilized prior to use. Incubation is typically performed under suitable conditions such as suitable temperature, osmolarity, aeration, agitation, etc. A person skilled in the art is aware of suitable incubation conditions for supporting or maintaining the growth/culturing of cells.

A “cell culture medium” (synonymously used: “culture medium”) according to the present invention is any mixture of components which maintains and/or supports the in vitro growth of cells and/or supports a particular physiological state. It is a chemically defined medium. The cell culture medium can comprise all components necessary to maintain and/or support the in vitro growth of cells or be used for the addition of selected components in combination with further components that are added separately.

Preferably the cell culture medium comprises all components necessary to maintain and/or support the in vitro growth of cells.

“Anaerobic condition” and “anaerobiosis” is defined as the maintenance of a substantially oxygen-free culture condition.

“Fresh culture medium” refers to any culture medium for microorganism growth or fermentation that has not been previously used, as a culture medium containing integrally all of its components.

“Stock or storage culture” refers to a fraction of microorganism in freezing medium stored for a determined period of time.

“Operational taxonomic unit (OTU, plural OTUs)” refers to a terminal leaf in a phylogenetic tree and is defined by a specific genetic sequence and all sequences that share sequence identity to this sequence at the level of species. A “type” or a plurality of “types” of bacteria includes an OTU or a plurality of different OTUs, and also encompasses a strain, species, genus, family or order of bacteria. The specific genetic sequence may be the 16S sequence or a portion of the 16S sequence or it may be a functionally conserved housekeeping gene found broadly across the eubacterial kingdom. OTUs share at least 95%, 96%, 97%, 98%, or 99% sequence identity. OTUs are frequently defined by comparing sequences between organisms. Sequences with less than 95% sequence identity are not considered to form part of the same OTU.

In microbiology, “16S sequencing” or “16S rRNA” or “16S-rRNA” or “16S” refers to sequence derived by characterizing the nucleotides that comprise the 16S ribosomal RNA gene(s). The bacterial 16S rDNA is approximately 1500 nucleotides in length and is used in reconstructing the evolutionary relationships and sequence similarity of one bacterial isolate to another using phylogenetic approaches. 16S sequences are used for phylogenetic reconstruction as they are in general highly conserved, but contain specific hypervariable regions that harbor sufficient nucleotide diversity to differentiate genera and species of most bacteria, as well as fungi.

The “V1-V9 regions” of the 16S rRNA refers to the first through ninth hypervariable regions of the 16S rRNA gene that are used for genetic typing of bacterial samples. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. Brosius et al., Complete nucleotide sequence of a 16S ribosomal RNA gene from Escherichia coli, PNAS 75(10):4801-4805 (1978). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU. A person of ordinary skill in the art can identify the specific hypervariable regions of a candidate 16S rRNA (in SEQ ID NOs. 1-1,4) by comparing the candidate sequence in question to the reference sequence and identifying the hypervariable regions based on similarity to the reference hypervariable regions.

### DESCRIPTION OF THE INVENTION

The cell culture media according to the present invention are designed to be suitable to grow or maintain/support the growth of one or more bacterial strains belonging to the genus Clostridium. Examples of Clostridium strains which growth is maintained/supported by the media according to the present invention are one or more strains listed in Tables 1 and 10.

Note that, the cluster of “bacteria belonging to the genus Clostridium” can be identified, for example, as follows. Specifically, the bacteria belonging to the genus Clostridium are classified by PCR using a primer set consisting of SEQ ID NOs 18 and 19 (for Clostridium spp. belonging to the cluster XIVa) or a primer set consisting of SEQ ID NOs 20 and 21 (for Clostridium spp. belonging to the cluster IV). In addition, the bacteria belonging to the genus Clostridium are classified by sequencing of 16S rRNA gene amplified using a primer set consisting of SEQ ID NOs 22 and 23.

SEQ ID NO: 18
AAATGACGGTACCTGACTAA
SEQ ID NO: 19
CTTTGAGTTTCATTCTTGCGAA
SEQ ID NO: 20
GCACAAGCAGTGGAGT
SEQ ID NO: 21
CTTCCTCCGTTTTGTCAA
SEQ ID NO: 22
AGAGTTTGATCMTGGCTCAG
SEQ ID NO: 23
ATTACCGCGGCKGCTG

The cell culture media, especially the full media, according to the present invention typically comprise at least one or more saccharide components, optionally one or more amino acids, optionally one or more vitamins or vitamin precursors, one or more salts, optionally one or more buffer components, optionally one or more co-factors, and optionally one or more nucleic acid components.

The media may also comprise sodium pyruvate, insulin, vegetable proteins, fatty acids and/or fatty acid derivatives and/or pluronic acid and/or surface active components like chemically prepared non-ionic surfactants.

Saccharide components are all mono- or di-saccharides, like glucose, galactose, ribose or fructose (examples of monosaccharides) or sucrose, lactose or maltose (examples of disaccharides).

Examples of amino acids according to the invention are tyrosine, the proteinogenic amino acids, especially the essential amino acids, leucine, isoleucine, lysine, methionine, phenylalanine, threonine, tryptophane and valine, as well as the non-proteinogenic amino acids like D-amino acids.

Examples of vitamins are Vitamin A (Retinol, retinal, various retinoids, and four carotenoids), Vitamin B1 (Thiamine), Vitamin B2 (Riboflavin), Vitamin B3 (Niacin, niacinamide), Vitamin B5 (Pantothenic acid), Vitamin B6 (Pyridoxine, pyridoxamine, pyridoxal), Vitamin B7 (Biotin), Vitamin B9 (Folic acid, folinic acid), Vitamin B12 (Cyanocobalamin, hydroxycobalamin, methylcobalamin), Vitamin C (Ascorbic acid), Vitamin D (Ergocalciferol, cholecalciferol), Vitamin E (Tocopherols, tocotrienols) and Vitamin K (phylloquinone, menaquinones). Vitamin precursors are also included.

Examples of salts are components comprising inorganic ions such as bicarbonate, calcium, chloride, magnesium, phosphate, potassium and sodium or trace elements such as Co, Cu, F, Fe, Mn, Mo, Ni, Se, Si, Ni, Bi, V and Zn. Examples are Copper(II) sulphate pentahydrate (CuSO4.5H2O), Sodium Chloride (NaCl), Calcium chloride (CaCl2·2H2O), Potassium chloride (KCl), Iron(II)sulphate, sodium phosphate monobasic anhydrous (NaH2PO4), Magnesium sulphate anhydrous (MgSO4), sodium phosphate dibasic anhydrous (Na2HPO4), Magnesium chloride hexahydrate (MgCl2·6H2O), zinc sulphate heptahydrate.

Examples of buffers are CO2/HCO3 (carbonate), phosphate, HEPES, PIPES, ACES, BES, TES, MOPS and TRIS.

Examples of cofactors are thiamine derivatives, biotin, vitamin C, NAD/NADP, cobalamin, flavin mononucleotide and derivatives, glutathione, heme nucleotide phosphates and derivatives.

Cells may be cultured in a variety of vessels including, for example, perfusion bioreactors, cell bags, culture plates, flasks and other vessels well known to those of ordinary skill in the art. Ambient conditions suitable for cell culture, such as temperature and atmospheric composition, are also well known to those skilled in the art. Methods for the culture of cells are also well known to those skilled in the art.

In one aspect the invention provides a composition, comprising the following components in the following amounts:
between 6.5-40 g/kg of Proteose Peptone (vegetable),
between 1-50 g/kg of Yeast extract,
between 5-15 g/kg of sodium phosphate dibasic (Na2HPO4),
between 1-50 g/kg of a sugar, and optionally
between 0.3-10 g/kg of a reducing agent, selected from the group consisting of sodium thioglycolate, L-cystein, and ascorbic acid.

In certain embodiments, the sugar can be a monosaccharide. In certain embodiments, the monosaccharide can be glucose or fructose. In certain embodiments, the sugar can be a disaccharide. In certain embodiments, the disaccharide can be sucrose. In certain embodiments, the sugar can be a trisaccharide. In certain embodiments, the trisaccharide can be raffinose.

In certain embodiments, the composition of the invention can comprise about 6.5 to about 8, about 7.5 to about 9, about 8 to about 10, about 9 to about 11, about 10 to about 12, about 11 to about 13, about 12 to about 14, about 13 to about 15, about 14 to about 16, about 15 to about 17, about 16 to about 18, about 17 to about 19, about 18 to about 20, about 19 to about 21, about 20 to about 22, about 21 to about 23, about 22 to about 24, about 23 to about 25, about 24 to about 26, about 25 to about 27, about 26 to about 28, about 27 to about 29, about 28 to about 30, about 29 to about 31, about 30 to about 32, about 31 to about 33, about 32 to about 34, about 33 to about 35, about 34 to about 36, about 35 to about 37, about 36 to about 38, about 37 to about 39, about 38 to about 40, about 39 to about 41, about 40 to about 42 g/kg of the Proteose Peptone (vegetable).

In certain embodiments, the composition of the invention can comprise about 0.3 to about 1, about 0.5 to about 1.5, about 1 to about 2, about 1.5 to about 2.5, about 2 to about 3, about 2.5 to about 3.5, about 3 to about 4, about 3.5 to about 4.5, about 4 to about 5, about 4.5 to about 5.5, about 5 to about 6, about 5.5 to about 6.5, about 6 to about 7, about 0.5 to about 2, about 0.8 to about 1.3 g/kg of a reducing agent. In certain embodiments the reducing agent is Sodium thioglycolate. In certain embodiments the reducing agent is L-cystein. In certain embodiments the reducing agent is ascorbic acid.

In certain embodiments, the composition of the invention can comprise about 1 to about 4, about 2 to about 5, about 3 to about 6, about 4 to about 7, about 5 to about 8, about 6 to about 9, about 7 to about 10, about 8 to about 11, about 8 to about 12, about 8 to about 13, about 8 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40 g/kg of Yeast extract.

In certain embodiments, the composition of the invention can comprise about 5 to about 10, about 6 to about 11, about 7 to about 12, about 8 to about 13, about 9 to about 14, about 10 to about 15 g/kg of sodium phosphate dibasic.

In certain embodiments, the composition of the invention can comprise about 1 to about 8, about 2 to about 9, about 3 to about 10, about 4 to about 11, about 5 to about 12, about 6 to about 13, about 7 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40, about 8 to about 45, about 8 to about 50, about 8 to about 55 g/kg of glucose.

In certain embodiments, the composition of the invention can comprise about 1 to about 8, about 2 to about 9, about 3 to about 10, about 4 to about 11, about 5 to about 12, about 6 to about 13, about 7 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40, about 8 to about 45, about 8 to about 50, about 8 to about 55 g/kg of sucrose.

In certain embodiments, the composition of the invention can comprise about 1 to about 8, about 2 to about 9, about 3 to about 10, about 4 to about 11, about 5 to about 12, about 6 to about 13, about 7 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40, about 8 to about 45, about 8 to about 50, about 8 to about 55 g/kg of raffinose.

In certain embodiments, the composition of the invention can comprise about 1 to about 8, about 2 to about 9, about 3 to about 10, about 4 to about 11, about 5 to about 12, about 6 to about 13, about 7 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40, about 8 to about 45, about 8 to about 50, about 8 to about 55 g/kg of fructose.

In one aspect the invention provides a composition, comprising the following components in the following amounts:
5-15 g/kg of Proteose Peptone,
5-15 g/kg of Phytone Peptone,
15-25 g/kg of Yeast extract,
9-14 g/kg of M9 salts,
5-15 g/kg of sugar,
optionally 2-6 g/kg of fatty acids, and
optionally 2-6 g/kg of polysorbate 20.

In certain embodiments, the sugar can be a monosaccharide. In certain embodiments, the monosaccharide can be glucose or fructose. In certain embodiments, the sugar can be a disaccharide. In certain embodiments, the disaccharide can be sucrose. In certain embodiments, the sugar can be a trisaccharide. In certain embodiments, the trisaccharide can be raffinose.

In certain embodiments, the composition of the invention can comprise about 5 to about 10, about 6 to about 10, about 8 to about 10, about 9 to about 11, about 10 to about 12, about 10 to about 13, about 10 to about 14, about 10 to about 15, about 10 to about 16, about 10 to about 17, about 10 to about 18, about 10 to about 19, about 10 to about 20, about 8 to about 12, about 8 to about 13, about 7 to about 12 g/kg of the Proteose Peptone.

In certain embodiments, the composition of the invention can comprise about 5 to about 10, about 6 to about 10, about 8 to about 10, about 9 to about 11, about 10 to about 12, about 10 to about 13, about 10 to about 14, about 10 to about 15, about 10 to about 16, about 10 to about 17, about 10 to about 18, about 10 to about 19, about 10 to about 20, about 8 to about 12, about 8 to about 13, about 7 to about 12 g/kg of the Phytone Peptone.

In certain embodiments, the composition of the invention can comprise about 15 to about 20, about 16 to about 20, about 17 to about 20, about 18 to about 20, about 18 to about 21, about 18 to about 22, about 18 to about 23, about 18 to about 24, about 18 to about 25, about 19 to about 20, about 19 to about 21, about 19 to about 22, about 19 to about 23, about 19 to about 24, about 19 to about 25, about 20 to about 25, about 20 to about 26, about 20 to about 27, about 20 to about 28, about 20 to about 29, about 20 to about 30 g/kg of Yeast extract.

M9 Salts is a well known in the art composition of salts, used in bacterial growth media (Sambrook, Fritsch and Maniatis. 1989. Molecular cloning: a laboratory manual, 2nd ed. Cold Spring Harbor Laboratory, Cold Spring Harbor, N.Y.). The M9 salts of the present invention contains:
33.9 g/L Na₂HPO₄
15 g/L KH₂PO₄
5 g/L NH₄Cl
2.5 g/L NaCl.

In certain embodiments, the composition of the invention can comprise about 9 to about 11.5, about 9.5 to about 11.5, about 10 to about 11.5, about 10.5 to about 11.5, about 11 to about 11.5, about 11 to about 12, about 11 to about 12.5, about 11 to about 13, about 11 to about 13.5, about 11 to about 12, about 11 to about 12.5, about 11 to about 13, about 11 to about 13.5, about 11 to about 14 g/kg of M9 salts.

In certain embodiments, the composition of the invention can comprise about 1 to about 8, about 2 to about 9, about 3 to about 10, about 4 to about 11, about 5 to about 12, about 6 to about 13, about 7 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40, about 8 to about 45, about 8 to about 50, about 8 to about 55, about 9 to about 11, about 9 to about 12, about 9 to about 13 g/kg of glucose.

In certain embodiments, the composition of the invention can comprise about 1 to about 8, about 2 to about 9, about 3 to about 10, about 4 to about 11, about 5 to about 12, about 6 to about 13, about 7 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40, about 8 to about 45, about 8 to about 50, about 8 to about 55, about 9 to about 11, about 9 to about 12, about 9 to about 13 g/kg of fructose.

In certain embodiments, the composition of the invention can comprise about 1 to about 8, about 2 to about 9, about 3 to about 10, about 4 to about 11, about 5 to about 12, about 6 to about 13, about 7 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40, about 8 to about 45, about 8 to about 50, about 8 to about 55, about 9 to about 11, about 9 to about 12, about 9 to about 13 g/kg of raffinose.

In certain embodiments, the composition of the invention can comprise about 1 to about 8, about 2 to about 9, about 3 to about 10, about 4 to about 11, about 5 to about 12, about 6 to about 13, about 7 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40, about 8 to about 45, about 8 to about 50, about 8 to about 55, about 9 to about 11, about 9 to about 12, about 9 to about 13 g/kg of sucrose.

As used herein, the term “fatty acids” is art recognized and includes a long-chain hydrocarbon based carboxylic acid. Fatty acids are components of many lipids including glycerides. The most common naturally occurring fatty acids are monocarboxylic acids which have an even number of carbon atoms (16 or 18) and which may be saturated or unsaturated. “Unsaturated” fatty acids contain cis double bonds between the carbon atoms. “Polyunsaturated” fatty acids contain more than one double bond and the double bonds are arranged in a methylene interrupted system (—CH═CH—CH2—CH═CH—).

In certain embodiments, the composition of the invention can comprise about 2 to about 4, about 2.5 to about 4, about 3 to about 4, about 2 to about 5, about 2.5 to about 5, about 3 to about 5, about 3.5 to about 5, about 3 to about 6, about 3.5 to about 6, about 4 to about 6, about 4 to about 5 g/kg of fatty acids.

In certain embodiments, the composition of the invention can comprise about 2 to about 4, about 2.5 to about 4, about 3 to about 4, about 2 to about 5, about 2.5 to about 5, about 3 to about 5, about 3.5 to about 5, about 3 to about 6, about 3.5 to about 6, about 4 to about 6, about 4 to about 5 g/kg of polysorbate 20.

In one aspect the invention provides a composition, comprising the following components in the following amounts:
45-55 g/kg of HY-Peptone,
15-25 g/kg of Yeast extract,
9-14 g/kg of M9 salts,
5-15 g/kg of sugar, and
optionally 2-6 g/kg of fatty acids.

In certain embodiments, the sugar can be a monosaccharide. In certain embodiments, the monosaccharide can be glucose or fructose. In certain embodiments, the sugar can be a disaccharide. In certain embodiments, the disaccharide can be sucrose. In certain embodiments, the sugar can be a trisaccharide. In certain embodiments, the trisaccharide can be raffinose.

In certain embodiments, the composition of the invention can comprise about 45 to about 50, about 46 to about 50, about 47 to about 50, about 47 to about 51, about 47 to about 52, about 47 to about 53, about 47 to about 54, about 47 to about 55, about 48 to about 55, about 49 to about 55, about 50 to about 55 g/kg of HY-Peptone.

In certain embodiments, the composition of the invention can comprise about 15 to about 20, about 16 to about 20, about 17 to about 20, about 18 to about 20, about 18 to about 21, about 18 to about 22, about 18 to about 23, about 18 to about 24, about 18 to about 25, about 19 to about 20, about 19 to about 21, about 19 to about 22, about 19 to about 23, about 19 to about 24, about 19 to about 25, about 20 to about 25, about 20 to about 26, about 20 to about 27, about 20 to about 28, about 20 to about 29, about 20 to about 30 g/kg of Yeast extract.

In certain embodiments, the composition of the invention can comprise about 9 to about 11.5, about 9.5 to about 11.5, about 10 to about 11.5, about 10.5 to about 11.5, about 11 to about 11.5, about 11 to about 12, about 11 to about 12.5, about 11 to about 13, about 11 to about 13.5, about 11 to about 12, about 11 to about 12.5, about 11 to about 13, about 11 to about 13.5, about 11 to about 14 g/kg of M9 salts.

In certain embodiments, the composition of the invention can comprise about 1 to about 8, about 2 to about 9, about 3 to about 10, about 4 to about 11, about 5 to about 12, about 6 to about 13, about 7 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40, about 8 to about 45, about 8 to about 50, about 8 to about 55, about 9 to about 11, about 9 to about 12, about 9 to about 13 g/kg of glucose.

In certain embodiments, the composition of the invention can comprise about 1 to about 8, about 2 to about 9, about 3 to about 10, about 4 to about 11, about 5 to about 12, about 6 to about 13, about 7 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40, about 8 to about 45, about 8 to about 50, about 8 to about 55, about 9 to about 11, about 9 to about 12, about 9 to about 13 g/kg of fructose.

In certain embodiments, the composition of the invention can comprise about 1 to about 8, about 2 to about 9, about 3 to about 10, about 4 to about 11, about 5 to about 12, about 6 to about 13, about 7 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40, about 8 to about 45, about 8 to about 50, about 8 to about 55, about 9 to about 11, about 9 to about 12, about 9 to about 13 g/kg of raffinose.

In certain embodiments, the composition of the invention can comprise about 1 to about 8, about 2 to about 9, about 3 to about 10, about 4 to about 11, about 5 to about 12, about 6 to about 13, about 7 to about 14, about 8 to about 15, about 8 to about 16, about 8 to about 17, about 8 to about 18, about 8 to about 19, about 8 to about 20, about 8 to about 21, about 8 to about 22, about 8 to about 23, about 8 to about 24, about 8 to about 25, about 8 to about 30, about 8 to about 35, about 8 to about 40, about 8 to about 45, about 8 to about 50, about 8 to about 55, about 9 to about 11, about 9 to about 12, about 9 to about 13 g/kg of sucrose.

As used herein, the term “fatty acids” is art recognized and includes a long-chain hydrocarbon based carboxylic acid. Fatty acids are components of many lipids including glycerides. The most common naturally occurring fatty acids are monocarboxylic acids which have an even number of carbon atoms (16 or 18) and which may be saturated or unsaturated. “Unsaturated” fatty acids contain cis double bonds between the carbon atoms. “Polyunsaturated” fatty acids contain more than one double bond and the double bonds are arranged in a methylene interrupted system (—CH═CH—CH2—CH═CH—).

In certain embodiments, the composition of the invention can comprise about 2 to about 4, about 2.5 to about 4, about 3 to about 4, about 2 to about 5, about 2.5 to about 5, about 3 to about 5, about 3.5 to about 5, about 3 to about 6, about 3.5 to about 6, about 4 to about 6, about 4 to about 5 g/kg of one or more fatty acids.

The following examples illustrate the invention. These examples should not be construed as to limit the scope of this invention. The examples are included for purposes of illustration and the present invention is limited only by the claims.

### Example 1. Initial Growth Conditions of Clostridium Strains

This study addressed the optimization of growth media for growing the bacteria of Clostridium genus, with the goal to reach a final concentration of at least 1.0×10⁹ cells/ml.

The following Clostridium strains were used in the experiments (Table 1).

TABLE 1
List of Clostridium strains.

| | | OTU (16S rDNA |
|---|---|---|
| Strain | Identification | fragment) sequence |
| 1 | Clostridium sp. 14774 | SEQ ID NO: 1 |
| 3 | Lachnospiraceae 7 1 58FAA | SEQ ID NO: 2 |
| 4 | Hungatella hathewayi | SEQ ID NO: 3 |
| 6 | Blautia producta | SEQ ID NO: 4 |
| 7 | Clostridium bolteae | SEQ ID NO: 5 |
| 8 | Clostridiaceae JC118 | SEQ ID NO: 6 |
| 9 | Anaerostipes unclassified | SEQ ID NO: 7 |
| 13 | Anaerotruncus colihominis | SEQ ID NO: 8 |
| 14 | Ruminococcus sp. ID8 | SEQ ID NO: 9 |
| 15 | Clostridium asparagiforme | SEQ ID NO: 10 |
| 16 | Clostridium 7 3 54FAA | SEQ ID NO: 11 |
| 18 | Coprobacillus unclassified | SEQ ID NO: 12 |
| 21 | Eubacterium fissicatena | SEQ ID NO: 13 |
| 26 | Lachnospiraceae bacterium | SEQ ID NO: 14 |
| | 5_1_57FAA | |
| 27 | Clostridium_sp_KLE_1755 | SEQ ID NO: 15 |
| 28 | Clostridiales 1_7_47FAA | SEQ ID NO: 16 |
| 29 | Lachnospiraceae 3_1_57FAA | SEQ ID NO: 17 |

The culture medium (Medium A) was prepared based on modification of Duncan Strong (DS) Medium (Duncan and Strong, Appl. Microbiol., Vol. 16, 1968), using the following ingredients:
Medium A:
Proteose peptone (vegetable) (Fluka, cat #29185)—7.5 g/kg
Vegetable extract (Sigma, cat #49869-500G-F)—7.5 g/kg
Yeast extract (BD, cat #212750)—4 g/kg
Sodium thioglycolate (Sigma, cat #T0632)—1 g/kg
Sodium phosphate dibasic (Na2HPO4, Sigma, cat #S5136-500)—10 g/kg
D-(+)-Raffinose pentahydrate (Sigma, cat #83400-100 g)—4 g/kg
Starch from potato, soluble (Sigma, cat #S9765)—0.5 g/kg
Water (purified using MILLI-Q® system)—1 L

The medium was sterilized by autoclaving (121° C. during 15 minutes) or sterile filtration using a 0.22 μm filter. The medium was placed in an anaerobic cabinet for approximately 16 hours, prior to use.

Frozen stock cultures of Clostridium strains (Table 1) were allowed to thaw in an anaerobic chamber. 400 μl of each strain were inoculated into a separate flask with 25 ml of Medium A, and incubated at 37° C. for 15 h, under anaerobic conditions. The optical density at 600 nm (OD₆₀₀ₙₘ) was monitored, and the count of colony forming units (CFUs) was done at the maximum OD₆₀₀ₙₘ, using Tryptic Soy agar plates (Table 2).

TABLE 2
Number of colony forming units (CFUs), cells/ml

| Strain | CFU, cells/ml |
|---|---|
| 1 | 1.4 × 10⁸ |
| 3 | No growth |
| 4 | 2.7 × 10⁸ |
| 6 | 8.4 × 10⁸ |
| 7 | 6.8 × 10⁸ |
| 8 | No growth |
| 9 | 1.9 × 10⁸ |
| 13 | 5.9 × 10⁷ |
| 14 | No growth |
| 15 | 1.2 × 10⁹ |
| 16 | 1 × 10⁸ |
| 18 | 3.1 × 10⁸ |
| 21 | 1.9 × 10⁹ |
| 26 | 2.2 × 10⁸ |
| 27 | 6.7 × 10⁸ |
| 28 | 4.6 × 10⁸ |
| 29 | 2 × 10⁸ |

With the aim to increase the growth yield, the experiments on optimization of growth media were carried out, using a Microbioreactor System (BIOLECTOR®, m2plabs). In BIOLECTOR®, the bacterial growth is continuously monitored by sending light of a defined wavelength into each well of a multiwell plate, whereas the backscattered light (indicator for biomass) is detected and analysed.

### Example 2. Effect of Carbon Source on Growth of Clostridium Strains

The original formulation for Medium A includes raffinose as the main carbon source. In order to find the best growth conditions, raffinose in Medium A was substituted with the same concentration of either sucrose (Sigma, cat #84100) or glucose (Sigma, cat #G5767), thus using one of the following compositions:
Proteose peptone (vegetable)—7.5 g/kg
Vegetable extract—7.5 g/kg
Yeast extract—4 g/kg
Sodium thioglycolate—1 g/kg
Sodium phosphate dibasic—10 g/kg
D-(+)-Raffinose pentahydrate, or D-(+)-Glucose, or D-Sucrose—4 g/kg
Starch—0.5 g/kg
Water (purified using MILLI-Q® system)—1 L

Initially, the frozen stock culture of each Clostridium strain (300 μL) was thawed and then inoculated in 3 mL of Medium A, containing 4 g/L of either raffinose, sucrose, glucose, or fructose, following by the incubation at 37° C. for 20 hours in the anaerobic chamber (termed pre-culture). Next, the OD₆₀₀ₙₘ of pre-culture was measured using the spectrophotometer, and the volume of pre-culture (X) to be inoculated into the culture, was calculated according to the formula:

Pre-culture volume X=Target OD₆₀₀ₙₘ(0.05)*Target Volume(1.4 ml)/Pre-culture OD₆₀₀ₙₘ

Next, the calculated volume of pre-culture was inoculated again into the total of 1.4 ml of Medium A, containing 4 g/L of either raffinose, sucrose, glucose, or fructose (termed culture) and was maintained at 37° C. during 48 hours, agitating at 600 rpm, under anaerobic conditions, using BIOLECTOR®.

The anaerobic conditions were achieved by using the mix of the following gases by volume: 10% of H2, 10% of CO2, and 80% of N₂. Alternatively, the following mix can be used: 5% of H2, 10% of CO2, and 85% of N₂.

The results showed that the preference for each carbon source was strain dependent (Table 3).

TABLE 3
Clostridium strains demonstrating faster
growth depending on the carbon source used.

| | Medium A | Medium A | Medium A | Medium A |
|---|---|---|---|---|
| Strain | (Raffinose) | (Fructose) | (Glucose) | (Sucrose) |
| 1 | + | ∘ | + | ++ |
| 3 | nd | ∘ | ∘ | ∘ |
| 4 | ++ | ++ | ++ | ++ |
| 6 | ++ | ++ | ++ | ++ |
| 7 | ++ | + | ++ | ++ |
| 8 | ∘ | ∘ | ∘ | ∘ |
| 9 | ∘ | + | + | ∘ |
| 13 | ∘ | ∘ | + | ∘ |
| 14 | + | + | ++ | ∘ |
| 15 | ++ | nd | nd | nd |
| 16 | nd | + | ++ | ∘ |
| 18 | ∘ | + | ++ | ++ |
| 21 | ++ | nd | nd | nd |
| 26 | ∘ | + | + | + |
| 27 | ++ | ++ | ++ | ++ |
| 28 | ++ | ++ | ++ | ++ |
| 29 | ++ | ++ | ++ | ++ |

Growth of the different strains was scored by the Biolector scattered light reading (Gain 40) after 20 h cultivation.
++—scattered light reading > 100;
+—20 h scattered light reading > 50;
∘—scattered light reading < 50;
nd—non-detectable.

### Example 3. Effect of Various Concentrations of Vegetable Extract, Peptone, Yeast Extract, and Carbon Source on Growth of Clostridium Strains

Next, Medium A was further optimized by increasing the concentrations of vegetable extract, peptone, yeast extract and/or carbon source, see Table 4.

TABLE 4
Various concentrations of vegetable extract, peptone,
yeast extract, and carbon source in Medium A.

| | Medium | Medium | Medium | Medium |
|---|---|---|---|---|
| Component | A1 | A2 | A3 | A4 |
| Proteose peptone, g/kg | 15 | 7.5 | 15 | 37.5 |
| Vegetable extract, g/kg | 15 | 7.5 | 15 | 37.5 |
| Yeast extract, g/kg | 8 | 4 | 8 | 20 |
| Na thyoglicolate, g/kg | 1 | 1 | 1 | 1 |
| Na₂PO₄, g/kg | 10 | 10 | 10 | 10 |
| Raffinose, or glucose, | 4 | 8 | 8 | 20 |
| or sucrose, g/kg | | | | |
| Starch, g/kg | 0.5 | 0.5 | 0.5 | 0.5 |

The growth curves for the Clostridium strains from Table 1 were analyzed using BIOLECTOR®, as described in Example 2. The results suggested that the bacterial growth was improved when Media A1, A2, or A3 was used. No benefit was observed when Medium A4 was used.

### Example 4. Effect of Starch and Vegetable Extract on Growth of Clostridium Strains

Next, Medium A was further optimized by removing starch from the recipe in Example 1, thus using the following composition:
Proteose peptone (vegetable)—7.5 g/kg
Vegetable extract #2—7.5 g/kg
Yeast extract—4 g/kg
Sodium thioglycolate—1 g/kg
Sodium phosphate dibasic—10 g/kg
Carbon source (strain dependent, see Table 3)—4 g/kg
Water (purified using MILLI-Q® system)—1 L

The growth curves for the Clostridium strains from Table 1 were analyzed using BIOLECTOR®, as described in Example 2. The results suggested that removal of starch did not have a significant effect on the growth curves.

Next, Medium A was further optimized by removing vegetable extract from the recipe in Example 1, thus using the following composition:
Proteose peptone (vegetal)—7.5 g/kg
Yeast extract—4 g/kg
Sodium thioglycolate—1 g/kg
Sodium phosphate dibasic—10 g/kg
Carbon source (strain dependent, see Table 3)—4 g/kg
Starch—0.5 g/kg
Water (purified using MILLI-Q® system)—1 L

The growth curves for the Clostridium strains from Table 1 were analyzed using BIOLECTOR®, as described in Example 2. The removal of vegetable extract did not have a significant effect on the growth curves.

### Example 5. Effect of Sodium Thioglycolate on Growth of Clostridium Strains

Next, Medium A was further optimized by removing Sodium thioglycolate from the recipe in Example 1, thus using the following composition:
Proteose peptone (vegetal)—7.5 g/kg
Vegetable extract—7.5 g/kg
Yeast extract—4 g/kg
Sodium phosphate dibasic—10 g/kg
Carbon source (strain dependent, see Table 3)—4 g/kg
Starch—0.5 g/kg
Water (purified using MILLI-Q® system)—1 L

The growth curves for the Clostridium strains from Table 1 were analyzed using BIOLECTOR®, as described in Example 2. The removal of Sodium thioglycolate had a significant growth inhibitory effect on strains 1, 4, 6, 7, 9, 18, 26, 27 and 28.

### Example 6. Effect of pH on Growth of Clostridium Strains

Next, Medium A, containing raffinose as the source of carbon, was further optimized by adjusting the pH to either 5.8, 6.3, or 7 using 100 mM sodium phosphate buffer.

The growth curves for the Clostridium strains from Table 1 were analyzed using BIOLECTOR®, as described in Example 2. The results suggested that there was a negative effect of lower pH on the growth rates (Table 5).

TABLE 5
Growth of Clostridium strains at different pH after 20 h.

| Strain | pH 5.8 | pH 6.3 | pH 7 |
|---|---|---|---|
| 1 | ∘ | ∘ | + |
| 3 | ∘ | ∘ | ∘ |
| 4 | ∘ | ++ | ++ |
| 6 | ∘ | ++ | ++ |
| 7 | + | ++ | ++ |
| 8 | ∘ | ∘ | ∘ |
| 9 | ∘ | ∘ | ∘ |
| 13 | ∘ | ∘ | ∘ |
| 14 | + | ++ | ++ |
| 16 | ∘ | ∘ | ∘* |
| 18 | ∘ | ∘ | ∘ |
| 26 | ∘ | ∘ | ∘ |
| 27 | ∘ | ∘ | ++ |
| 28 | + | ++ | ++ |
| 29 | ∘ | + | + |

Growth of the different strains was scored by the Biolector scattered light reading (Gain 40) after 20 h cultivation.
++—scattered light reading > 100;
+—20 h scattered light reading > 50;
∘—scattered light reading < 50.
*While all scattered light readings were below the threshold, the scattered light signal for strain 26, pH 7 was substantially higher than for the other pHs.

### Example 7. Effect of Antifoam on Growth of Clostridium Strains

In order to prevent the formation of foam during the exponential phase of the bacterial growth, Antifoam 204 (100 μL/L, Sigma) was added to the Medium, thus using the following composition:
Proteose peptone (vegetal)—7.5 g/kg
Vegetable extract—7.5 g/kg
Yeast extract—4 g/kg
Sodium thioglycolate—1 g/kg
Sodium phosphate dibasic—10 g/kg
Carbon source (strain dependent, see Table 3)—4 g/kg
Starch—0.5 g/kg
Water (purified using MILLI-Q® system)—1 L

The growth curves for the Clostridium strains from Table 1 were analyzed using BIOLECTOR®, as described in Example 2. The results suggested that the addition of antifoam had no negative effect on the bacterial growth rates.

### Example 8. Effect of the Basic Medium on Growth of Clostridium Strains

The final media includes all changes described above and was named Basic Medium (BM, Table 6). The added sugar in the medium was strain dependent, so the final media were termed “BMR” if it comprised raffinose, “BMS” is it comprised sucrose, or “BMG” if it comprised glucose:

TABLE 6
Optimized media compositions (termed Basic Media, or BM).

| | | | BMS | | | |
|---|---|---|---|---|---|---|
| | | | (Strains 1, 4, 6, | | BMG | |
| | BMR | | 7, 9, 15, 21, | | (Strains 13, | |
| Component | (strain 27) | | 26, 28, 29) | | 14, 16) | |
| Proteose peptone | 30 | g/kg | 30 | g/kg | 30 | g/kg |
| (vegetable) | | | | | | |
| Yeast extract | 8 | g/kg | 8 | g/kg | 8 | g/kg |
| Sodium thioglycolate | 1 | g/kg | 1 | g/kg | 1 | g/kg |
| Na₂HPO₄ | 10 | g/kg | 10 | g/kg | 10 | g/kg |
| D-(+)-Glucose | — | | — | | 8 | g/kg |
| D-(+)-Raffinose | 8 | g/kg | — | | — | |
| pentahydrate | | | | | | |
| D-Sucrose | — | | 8 | g/kg | — | |
| Antifoam 204 | 100 | μL/kg | 100 | μL/kg | 100 | μL/kg |

A comparison between Medium A and BM was carried out for Clostridium strains (Table 7).

TABLE 7
The growth of Clostridium strains in Medium A and BM medium.

| | | Sugar in | Medium A | Medium A | Medium A | Medium A |
|---|---|---|---|---|---|---|
| Strain | BM | BM | (Raffinose) | Fructose | Glucose | Sucrose |
| 1 | ++ | Sucrose | + | o | + | ++ |
| 3 | nd | | nd | o | o | o |
| 4 | ++ | Sucrose | ++ | ++ | ++ | ++ |
| 6 | ++ | Sucrose | ++ | ++ | ++ | ++ |
| 7 | ++ | Sucrose | ++ | + | ++ | ++ |
| 8 | nd | | o | o | o | o |
| 9 | ++ | Sucrose | o | + | + | o |
| 13 | ++ | Glucose | o | o | + | o |
| 14 | ++ | Glucose | + | + | ++ | o |
| 15 | ++ | Sucrose | ++ | nd | nd | nd |
| 16 | ++ | Glucose | nd | + | ++ | o |
| 18 | ++ | Sucrose | o | + | ++ | ++ |
| 21 | ++ | Sucrose | ++ | nd | nd | nd |
| 26 | ++ | Sucrose | o | + | + | ++ |
| 27 | ++ | Raffinose | ++ | ++ | ++ | ++ |
| 28 | ++ | Sucrose | ++ | ++ | ++ | ++ |
| 29 | ++ | Sucrose | ++ | ++ | ++ | ++ |

Growth of the different strains was scored by the Biolector scattered light reading (Gain 40) after 20 h cultivation.
++—scattered light reading >100;
+—20 h scattered light reading >50;
o—scattered light reading <50;
nd—non-detectable.

### Example 9. Effect of the High-Purity Peptone on the Growth of Clostridium Strains

A high-purity degree peptone (VEGETABLE PEPTONE No 1, OXOID, VG0100), in a concentration 30 g/kg, was tested instead the original Proteose peptone in the media composition according to Table 5. The growth curves for the Clostridium strains from Table 1 were analyzed using BIOLECTOR®, as described in Example 2. The results suggested that there was a negative impact of this peptone on the growth of bacteria of strains number 1, 4, 6, 9, 18 and 29.

### Example 10. Growth of Clostridium Strains Using M1 Medium

With the goal to develop one single medium to grow the 17 Clostridium strains to the CFU of approximately 10⁹ cells/ml, as well as to improve the growth of strain 3 and strain 8, a novel medium composition has been tested.
M1 medium:
Proteose Peptone (Fluka, cat #29185-500F)—10 g/kg
Phytone peptone (BD, cat #211906)—10 g/kg
Yeast extract (BD, cat #212730)—10 g/kg
M9 salts (33.9 g/L Na2HPO4, 15 g/L KH2PO4, 5 g/L NH4Cl, 2.5 g/L NaCl, BD, cat #248510)—11.4 g/kg
D-(+)-Glucose (Sigma, cat #G5767)—10 g/kg
Trace elements metal mix cocktail powder (Sigma SAFC, 87148CP)—0.1 g/kg
Fatty acids (Sigma SAFC, cat #F7050)—4 g/kg
Water (purified using MILLI-Q® R system)—1 L

The growth curves for the Clostridium strains from Table 1 were analyzed as described in Example 1. The results suggested that all Clostridium strains were growing well in M1 medium (Table 8). All strains except for strain 3 and strain 27 were observed to reach the desired CFU of 10′ cells/ml (Table 8).

TABLE 8
Number of colony forming units (CFUs), cells/ml,
of Clostridium cultures, grown in M1 medium.

| Strain | 24 hr | 30 hr | 48 hr | 72 hr |
|---|---|---|---|---|
| 3 | 2.6E+08 | 2.50E+08 | 2.0E+08 | 2.9E+08 |
| 1 | 3.92E+08 | 5.56E+08 | 7.88E+08 | 1.24E+09 |
| 4 | 2.40E+09 | 2.36E+09 | 2.28E+09 | 3.68E+09 |
| 13 | 3.68E+09 | 4.92E+09 | 5.92E+09 | 4.08E+09 |
| 14 | 8.24E+08 | 2.16E+09 | 2.64E+09 | 4.04E+09 |
| 18 | 1.04E+09 | 9.60E+08 | 9.20E+08 | 8.80E+08 |
| 27 | 3.48E+08 | 3.08E+08 | 3.40E+08 | 4.32E+08 |
| 6 | 3.56E+09 | 3.20E+09 | 3.68E+09 | 3.36E+09 |
| 7 | 3.64E+09 | 3.40E+09 | 4.68E+09 | 4.08E+09 |
| 15 | 9.60E+06 | 1.76E+08 | 3.96E+09 | 2.96E+09 |
| 21 | 1.60E+08 | 1.72E+09 | 1.17E+10 | 6.36E+09 |
| 26 | 3.64E+08 | 1.46E+09 | 8.28E+09 | 4.00E+09 |
| 28 | 7.76E+09 | 7.76E+09 | 7.72E+09 | 8.72E+09 |
| 29 | 4.76E+08 | 2.28E+08 | 2.04E+08 | 1.08E+08 |
| 8 | 1.7E+09 | 1.77E+09 | 3.2E+08 | 1.4E+09 |
| 9 | 6.68E+09 | 6.44E+09 | 5.90E+09 | 6.60E+09 |
| 16 | 3.72E+07 | 3.32E+08 | 4.86E+09 | 4.48E+09 |

Additional experiments were done to further optimize the M1 medium. Trace elements metal mix and Fatty acids were removed from the M1 media composition described above, and bacterial cell growth was tested. The results suggested that the bacterial growth was not impacted significantly.

### Example 11. Growth of Clostridium Strains Using M81 Medium

With the goal to develop one single medium to grow the 17 Clostridium strains to the CFU of approximately 10′ cells/ml, as well as to improve the growth of strain 3, the M1 medium was modified as described below, the new medium was termed M81:
M81 medium:
HY peptone (Kerry Biosciences, cat #5X01111) 50 g/kg
Yeast extract (BD, cat #212730)—10 g/kg
M9 salts (33.9 g/L Na2HPO4, 15 g/L KH2PO4, 5 g/L NH4Cl, 2.5 g/L NaCl, BD, cat #248510)—11.4 g/kg
D-(+)-Glucose (Sigma, cat #G5767)—10 g/kg
Trace elements metal mix cocktail powder (Sigma SAFC, 87148CP)—0.1 g/kg
Fatty acid (Sigma SAFC, cat #F7050)—4 g/kg
Water (purified using MILLI-Q® system)—1 L

The growth curves for the Clostridium strains from Table 1 were analyzed as described in Example 1. The results suggested that at least Clostridium strains #1, 3, 8, 18, 27, and 29 were growing well in M81 medium (Table 7). At least strains #3 and 18 were observed to reach the desired CFU of 10⁹ cells/ml (Table 9).

TABLE 9
Number of colony forming units (CFUs), cells/ml,
of Clostridium cultures, grown in M81 medium.

| Strain | 24 hr | 48 hr | 72 hr |
|---|---|---|---|
| 3 | 2.09E+07 | 1.10E+09 | 1.15E+09 |
| 1 | 3.84E+07 | 3.70E+08 | 8.80E+08 |
| 8 | 1.76E+08 | 4.96E+08 | n/a |
| 18 | 1.18E+09 | 1.30E+09 | 1.41E+09 |
| 27 | 8.92E+07 | 3.72E+08 | n/a |
| 29 | 1.72E+07 | 5.68E+08 | n/a |

Additional experiments were done to further optimize the M81 medium. Trace elements metal mix and Fatty acids were removed from the M81 medium composition described above, and bacterial cell growth was tested. The results suggested that the bacterial growth was not impacted significantly.

TABLE 10
OTU (16S rDNA fragment) sequences.

| SEQ | |
|---|---|
| ID | |
| NO: | Nucleotide sequence |
| 1 | CAGAATTAAGAAACACACGTCAAAGAAGGATCCGGAAACGGATAG |
| | AAGAGAAAAAGAAGAGCTAAACAAACGAAAGATTGTAAAGATAAA |
| | GAGACAATGGAGAGTTTGATCCTGGCTCAGGATGAACGCTGGCGG |
| | CGTGCCTAATACATGCAAGTCGAACGCGGGCAGCAATGCCCGAGT |
| | GGCGAACGGGTGAGTAATACATAAGTAACCTGCCCTTTACAGGGG |
| | GATAACTATTGGAAACGATAGCTAAGACCGCATAGGTAAAGATAC |
| | CGCATGGTAAGTTTATTAAAAGTGCCAAGGCACTGGTAGAGGATG |
| | GACTTATGGCGCATTAGCTAGTTGGTGAGGTAACGGCTCACCAAG |
| | GCGACGATGCGTAGCCGACCTGAGAGGGTGACCGGCCACACTGGG |
| | ACTGAGACACGGCCCAGACTCCTACGGGAGGCAGCAGTAGGGAAT |
| | TTTCGGCAATGGGGGGAACCCTGACCGAGCAACGCCGCGTGAAGG |
| | AGGAAGGTCTTCGGACTGTAAACTTCTGTTATAAAGGAAGAAAGG |
| | CGGATACAGGGAATGGTATCCGAGTGACGGTACTTTATGAGGAAG |
| | CCACGGCTAACTACGTGCCAGCAGCCGCGGTAATACGTAGGTGGC |
| | AAGCGTTATCCGGAATTATTGGGCGTAAAGAGGGAGCAGGCGGCA |
| | GCAAGGGTCTGTGGTGAAAGACTGAAGCTTAACTTCAGTAAGCCA |
| | TAGAAACCGGGCAGCTAGAGTGCAGGAGAGGATCGTGGAATTCCA |
| | TGTGTAGCGGTGAAATGCGTAGATATATGGAGGAACACCAGTGGC |
| | GAAGGCGACGATCTGGCCTGCAACTGACGCTCAGTCCCGAAAGCG |
| | TGGGGAGCAAATAGGATTAGATACCCTAGTAGTCCACGCCGTAAA |
| | CGATGAGTACTAAGTGTTGGGAGTCAAATCTCAGTGCTGCAGTTA |
| | ACGCAGTAAGTACTCCGCCTGAGTAGTACGTTCGCAAGAATGAAA |
| | CTCAAAGGAATTGACGGGGGCCCGCACAAGCGGTGGAGCATGTGG |
| | TTTAATTCGAAGCAACGCGAAGAACCTTACCAGGTCTTGACATAC |
| | TCATAAAGGCTCCAGAGATGGAGAGATAGATATATGGGATACAGG |
| | TGGTGCATGGTTGTCGTCAGCTCGTGTCGTGAGATGTTGGGTTAA |
| | GTCCCGCAACGAGCGCAACCCTTATCGTTAGTTACCATCATTAAG |
| | TTGGGGACTCTAGCGAGACTGCCAGTGACAAGCTGGAGGAAGGCG |
| | GGGATGACGTCAAATCATCATGCCCCTTATGACCTGGGCTACACA |
| | CGTGCTACAATGGATGGAGCAGAGGGAAGCGAAGCCGCGAGGTGG |
| | AGCGAAACCCAGAAAACCATTCTCAGTTCGGATTGTAGTCTGCAA |
| | CTCGACTACATGAAGTTGGAATCGCTAGTAATCGCGAATCAGCAT |
| | GTCGCGGTGAATACGTTCTCGGGCCTTGTACACACCGCCCGTCAC |
| | ACCATGAGAGTTGATAACACCCGAAGCCGGTGGCCTAACCGCAAG |
| | GAGGGAGCTGTCTAAGGTGGGATTGATGATTGGGGTGAAGTCGTA |
| | ACAAGGTATCCCTACGGGAACGTGGGGATGGATCACCTCCTTTCT |
| | AAGGAGAAAGAAGAGAAGTGAAGTTTCATTAATACTGTTTAGTTT |
| | TGAGTGATGCTCAGGAGG |
| 2 | TTAAGCTTCGATAAGATGATTTGAGGAACGGAGACGTTCTTTAGA |
| | TACCATTTTATTGAGAGTTTGATCCTGGCTCAGGATGAACGCTGG |
| | CGGCGTGCTTAACACATGCAAGTCGAACGGGGTGCTCATGACGGA |
| | GGATTCGTCCAACGGATTGAGTTACCTAGTGGCGGACGGGTGAGT |
| | AACGCGTGAGGAACCTGCCTTGGAGAGGGGAATAACACTCCGAAA |
| | GGAGTGCTAATACCGCATGATGCAGTTGGGTCGCATGGCTCTGAC |
| | TGCCAAAGATTTATCGCTCTGAGATGGCCTCGCGTCTGATTAGCT |
| | AGTAGGCGGGGTAACGGCCCACCTAGGCGACGATCAGTAGCCGGA |
| | CTGAGAGGTTGACCGGCCACATTGGGACTGAGACACGGCCCAGAC |
| | TCCTACGGGAGGCAGCAGTGGGGAATATTGGGCAATGGGCGCAAG |
| | CCTGACCCAGCAACGCCGCGTGAAGGAAGAAGGCTTTCGGGTTGT |
| | AAACTTCTTTTGTCGGGGACGAAACAAATGACGGTACCCGACGAA |
| | TAAGCCACGGCTAACTACGTGCCAGCAGCCGCGGTAATACGTAGG |
| | TGGCAAGCGTTATCCGGATTTACTGGGTGTAAAGGGCGTGTAGGC |
| | GGGATTGCAAGTCAGATGTGAAAACTGGGGGCTCAACCTCCAGCC |
| | TGCATTTGAAACTGTAGTTCTTGAGTGCTGGAGAGGCAATCGGAA |
| | TTCCGTGTGTAGCGGTGAAATGCGTAGATATACGGAGGAACACCA |
| | GTGGCGAAGGCGGATTGCTGGACAGTAACTGACGCTGAGGCGCGA |
| | AAGCGTGGGGAGCAAACAGGATTAGATACCCTGGTAGTCCACGCC |
| | GTAAACGATGGATACTAGGTGTGGGGGGTCTGACCCCCTCCGTGC |
| | CGCAGTTAACACAATAAGTATCCCACCTGGGGAGTACGATCGCAA |
| | GGTTGAAACTCAAAGGAATTGACGGGGGCCCGCACAAGCGGTGGA |
| | GTATGTGGTTTAATTCGAAGCAACGCGAAGAACCTTACCAGGGCT |
| | TGACATCCCACTAACGAAGCAGAGATGCATTAGGTGCCCTTCGGG |
| | GAAAGTGGAGACAGGTGGTGCATGGTTGTCGTCAGCTCGTGTCGT |
| | GAGATGTTGGGTTAAGTCCCGCAACGAGCGCAACCCTTATTGTTA |
| | GTTGCTACGCAAGAGCACTCTAGCGAGACTGCCGTTGACAAAACG |
| | GAGGAAGGTGGGGACGACGTCAAATCATCATGCCCCTTATGTCCT |
| | GGGCCACACACGTACTACAATGGTGGTTAACAGAGGGAGGCAATA |
| | CCGCGAGGTGGAGCAAATCCCTAAAAGCCATCCCAGTTCGGATTG |
| | CAGGCTGAAACCCGCCTGTATGAAGTTGGAATCGCTAGTAATCGC |
| | GGATCAGCATGCCGCGGTGAATACGTTCCCGGGCCTTGTACACAC |
| | CGCCCGTCACACCATGAGAGTCGGGAACACCCGAAGTCCGTAGCC |
| | TAACCGCAAGGAGGGCGCGGCCGAAGGTGGGTTCGATAATTGGGG |
| | TGAAGTCGTAACAAGGTAGCCGTATCGGAAGGTGCGGCTGGATCA |
| | CCTCCTTTCTAAGGAGACTTCAACGGAAGTAGTTTTCTGTTGTAA |
| | CGTCCTAAGGTCAGCTTTCTGGAGCGAATGGCTTTCACGTTGTTT |
| | AATTTAGAGGGTACACTCTC |
| 3 | ATTCAGAACGAATACAATTATTGTATACGAACCAAACAACAAGTA |
| | AAACGGGAAATAAATTAGCTAGTTAGTTGATTTTGACCCCGGATC |
| | AAACGAATCATTTAACATGAGAGTTCGATCCTGGCTCAGGATGAA |
| | CGCTGGCGGCGTGCTTAACACATGCAAGTCGAGCGAAGCGGTTTC |
| | AATGAAGTTTTCGGATGGAATTGAAATTGACTTAGCGGCGGACGG |
| | GTGAGTAACGCGTGGGTAACCTGCCTTACACTGGGGGATAACAGT |
| | TAGAAATGACTGCTAATACCGCATAAGCGCACAGGGCCGCATGGT |
| | CTGGTGTGAAAAACTCCGGTGGTGTAAGATGGACCCGCGTCTGAT |
| | TAGGTAGTTGGTGGGGTAACGGCCCACCAAGCCGACGATCAGTAG |
| | CCGACCTGAGAGGGTGACCGGCCACATTGGGACTGAGACACGGCC |
| | CAAACTCCTACGGGAGGCAGCAGTGGGGAATATTGGACAATGGGC |
| | GAAAGCCTGATCCAGCGACGCCGCGTGAGTGAAGAAGTATTTCGG |
| | TATGTAAAGCTCTATCAGCAGGGAAGAAAATGACGGTACCTGACT |
| | AAGAAGCCCCGGCTAACTACGTGCCAGCAGCCGCGGTAATACGTA |
| | GGGGGCAAGCGTTATCCGGATTTACTGGGTGTAAAGGGAGCGTAG |
| | ACGGTTAAGCAAGTCTGAAGTGAAAGCCCGGGGCTCAACCCCGGT |
| | ACTGCTTTGGAAACTGTTTGACTTGAGTGCAGGAGAGGTAAGTGG |
| | AATTCCTAGTGTAGCGGTGAAATGCGTAGATATTAGGAGGAACAC |
| | CAGTGGCGAAGGCGGCTTACTGGACTGTAACTGACGTTGAGGCTC |
| | GAAAGCGTGGGGAGCAAACAGGATTAGATACCCTGGTAGTCCACG |
| | CCGTAAACGATGAATACTAGGTGTCGGGGGACAACGTCCTTCGGT |
| | GCCGCCGCTAACGCAATAAGTATTCCACCTGGGGAGTACGTTCGC |
| | AAGAATGAAACTCAAAGGAATTGACGGGGACCCGCACAAGCGGTG |
| | GAGCATGTGGTTTAATTCGAAGCAACGCGAAGAACCTTACCAAGT |
| | CTTGACATCCCATTGAAAATCATTTAACCGTGATCCCTCTTCGGA |
| | GCAATGGAGACAGGTGGTGCATGGTTGTCGTCAGCTCGTGTCGTG |
| | AGATGTTGGGTTAAGTCCCGCAACGAGCGCAACCCTTATCCTTAG |
| | TAGCCAGCACATGATGGTGGGCACTCTGGGGAGACTGCCAGGGAT |
| | AACCTGGAGGAAGGTGGGGATGACGTCAAATCATCATGCCCCTTA |
| | TGATTTGGGCTACACACGTGCTACAATGGCGTAAACAAAGGGAAG |
| | CAAAGGAGCGATCTGGAGCAAACCCCAAAAATAACGTCTCAGTTC |
| | GGATTGCAGGCTGCAACTCGCCTGCATGAAGCTGGAATCGCTAGT |
| | AATCGCGAATCAGAATGTCGCGGTGAATACGTTCCCGGGTCTTGT |
| | ACACACCGCCCGTCACACCATGGGAGTTGGTAACGCCCGAAGTCA |
| | GTGACCCAACCGTAAGGAGGGAGCTGCCGAAGGCGGGACTGATAA |
| | CTGGGGTGAAGTCGTAACAAGGTAGCCGTATCGGAAGGTGCGGCT |
| | GGATCACCTCCTTTCTAAGGAAGAAGAAGTAAGGGTTTTATATAC |
| | TGTTGAGTCTTGTGTTTCAAAGGAAGGCGAAAGCCGAAGAAAAAT |
| | AGAATAAAAGAATAGAAACATCAGAGACAAAGAGATTCTGGTGCC |
| | GA |
| 4 | CGAAGTCTGGTGTGGAATATTCAGACACCCCAAATTTTTTCATAC |
| | ACTGCGATCCGGGAAGCATATGAGGAAGCGCGGCAGCAGAGCATG |
| | GAGGATATTACAGATGATGCCATGGTCATGGAGCGCTTTGGAAAC |
| | TTGAAAATTAAGTTGGTGGAAGGTTCCTATGAGAACATTAAAATC |
| | ACTACGCCGGAGGACATTCTTGTCGCTGAGAAAATTTTGGAAAAA |
| | AATTAGAGAAATCTATTGACAACTTCCGACATAAGTGATAAGATA |
| | CCACTTGTCGCTGACCGAGTGACAAACATGGAGAGATATCGAAGT |
| | GGTCATAACGAGGCGGTCTTGAAAACCGTTTGTCCGCAAGGGCGC |
| | GTGGGTTCGAATCCCACTCTCTCCGTTCCCGAAAGCAGATATCGA |
| | GAACGAATCAGAGTGACGAGTTCAAGATTAGTTATGATGCTTTTG |
| | GATATATAGAGAAGCAGTTATTTGAAAGCTTACTTTCAAACTGTT |
| | GTTTTATTCAGCCTGGAGAAGTACCCAAGCGGCTGAAGGGGCTCC |
| | CCTGGAAAGGGAGTAGGTCGTTAATAGCGGCGCGAGGGTTCAAAT |
| | CCCTCCTTCTCCGTTTTCTCTTAAAAAAAGAGAAAAAAGTTGTTG |
| | ACAAGCGATCGGATATATGATAATCTATTATAGCTGTCGCAAAAA |
| | CGACAACGATTTATGACATTGAAAATGAGCTTTGCTTAAAAGCTT |
| | TGTGAGTAAATGCAAAGGTTTAGCGTAGTTGAAAGAAGTTTCGAC |
| | ACAACGTAAAAAAACTTTTGAAAAACAATACAATTAAAAATAAAT |
| | TTTTAATTGTCATAAGTTCTTGACAAGCACGAAACGATATGATAA |
| | AATATCAAAGCTGTCAAAAACTGACAGGAACCTTGATAACTGAAC |
| | AGTGAAACACATGAATCGAAAATTCTTTTACATTCATTAGTAACA |
| | AAACGAACGGTTCTAAACGAACCAAAACAGTAAAAGGGATATCTT |
| | ATTGAGCCTAGCTCAATAAGCCAGAATTAGCCAAAGTTAATTCTG |
| | TTCCTGGACAAACACTTGATCAGAGAGTTTGATCCTGGCTCAGGA |
| | TGAACGCTGGCGGCGTGCTTAACACATGCAAGTCGAGCGAAGCAT |
| | TAAGACAGATTTCTTCGGATTGAAGTCTTTGTGACTGAGCGGCGG |
| | ACGGGTGAGTAACGCGTGGGTAACCTGCCTCATACAGGGGGATAA |
| | CAGTTAGAAATGACTGCTAATACCGCATAAGCGCACAGGAcCGCA |
| | TGGTCTGGTGTGAAAAACTCCGGTGGTATGAGATGGACCCGCGTC |
| | TGATTAGCTAGTTGGAGGGGTAACGGCCCACCAAGGCGACGATCA |
| | GTAGCCGGCCTGAGAGGGTGAACGGCCACATTGGGACTGAGACAC |
| | GGCCCAGACTCCTACGGGAGGCAGCAGTGGGGAATATTGCACAAT |
| | GGGGGAAACCCTGATGCAGCGACGCCGCGTGAAGGAAGAAGTATC |
| | TCGGTATGTAAACTTCTATCAGCAGGGAAGAAAATGACGGTACCT |
| | GACTAAGAAGCCCCGGCTAACTACGTGCCAGCAGCCGCGGTAATA |
| | CGTAGGGGGCAAGCGTTATCCGGATTTACTGGGTGTAAAGGGAGC |
| | GTAGACGGAAGAGCAAGTCTGATGTGAAAGGCTGGGGCTTAACCC |
| | CAGGACTGCATTGGAAACTGTTGTTCTAGAGTGCCGGAGAGGTAA |
| | GCGGAATTCCTAGTGTAGCGGTGAAATGCGTAGATATTAGGAGGA |
| | ACACCAGTGGCGAAGGCGGCTTACTGGACGGTAACTGACGTTGAG |
| | GCTCGAAAGCGTGGGGAGCAAACAGGATTAGATACCCTGGTAGTC |
| | CACGCCGTAAACGATGAATACTAGGTGTCGGGTGGCAAAGCCATT |
| | CGGTGCCGCAGCAAACGCAATAAGTATTCCACCTGGGGAGTACGT |
| | TCGCAAGAATGAAACTCAAAGGAATTGACGGGGACCCGCACAAGC |
| | GGTGGAGCATGTGGTTTAATTCGAAGCAACGCGAAGAACCTTACC |
| | AAGTCTTGACATCCCTCTGACCGTCCCGTAAtGGGGgCTTCCCTT |
| | CGGGGCAGAGGAGACAGGTGGTGCATGGTTGTCGTCAGCTCGTGT |
| | CGTGAGATGTTGGGTTAAGTCCCGCAACGAGCGCAACCCTTATCC |
| | TTAGTAGCCAGCACATGATGGTGGGCACTCTAGGGAGACTGCCGG |
| | GGATAACCCGGAGGAAGGCGGGGACGACGTCAAATCATCATGCCC |
| | CTTATGATTTGGGCTACACACGTGCTACAATGGCGTAAACAAAGG |
| | GAAGCGAGACAGCGATGTTGAGCGAATCCCAAAAATAACGTCCCA |
| | GTTCGGACTGCAGTCTGCAACTCGACTGCACGAAGCTGGAATCGC |
| | TAGTAATCGCGGATCAGAATGCCGCGGTGAATACGTTCCCGGGTC |
| | TTGTACACACCGCCCGTCACACCATGGGAGTCAGTAACGCCCGAA |
| | GTCAGTGACCTAACCGAAAGGAAGGAGCTGCCGAAGGCGGGACCG |
| | ATAACTGGGGTGAAGTCGTAACAAGGTAGCCGTATCGGAAGGTGC |
| | GGCTGGATCACCTCCTTTCTAAGGAAGAAGAAGTAGAGAAAAGTG |
| | TTTCACTGTTGAGTTACCAAGA |
| 5 | GAAAATTCAGAACAAAACCAAGTAATGAAGGTTAAAGATAACTAG |
| | CCAAGCTAAGTAGTCTGAAACCTGGAATCAACTTTTAAACATGAG |
| | AGTTTGATCCTGGCTCAGGATGAACGCTGGCGGCGTGCCTAACAC |
| | ATGCAAGTCGAACGAAGCAATTAAAATGAAGTTTTCGGATGGATT |
| | TTTGATTGACTGAGTGGCGGACGGGTGAGTAACGCGTGGATAACC |
| | TGCCTCACACTGGGGGATAACAGTTAGAAATGACTGCTAATACCG |
| | CATAAGCGCACAGTACCGCATGGTACGGTGTGAAAAACTCCGGTG |
| | GTGTGAGATGGATCCGCGTCTGATTAGCCAGTTGGCGGGGTAACG |
| | GCCCACCAAAGCGACGATCAGTAGCCGACCTGAGAGGGTGACCGG |
| | CCACATTGGGACTGAGACACGGCCCAAACTCCTACGGGAGGCAGC |
| | AGTGGGGAATATTGCACAATGGGCGAAAGCCTGATGCAGCGACGC |
| | CGCGTGAGTGAAGAAGTATTTCGGTATGTAAAGCTCTATCAGCAG |
| | GGAAGAAAATGACGGTACCTGACTAAGAAGCCCCGGCTAACTACG |
| | TGCCAGCAGCCGCGGTAATACGTAGGGGGCAAGCGTTATCCGGAT |
| | TTACTGGGTGTAAAGGGAGCGTAGACGGCGAAGCAAGTCTGAAGT |
| | GAAAACCCAGGGCTCAACCCTGGGACTGCTTTGGAAACTGTTTTG |
| | CTAGAGTGTCGGAGAGGTAAGTGGAATTCCTAGTGTAGCGGTGAA |
| | ATGCGTAGATATTAGGAGGAACACCAGTGGCGAAGGCGGCTTACT |
| | GGACGATAACTGACGTTGAGGCTCGAAAGCGTGGGGAGCAAACAG |
| | GATTAGATACCCTGGTAGTCCACGCCGTAAACGATGAATGCTAGG |
| | TGTTGGGGGGCAAAGCCCTTCGGTGCCGTCGCAAACGCAGTAAGC |
| | ATTCCACCTGGGGAGTACGTTCGCAAGAATGAAACTCAAAGGAAT |
| | TGACGGGGACCCGCACAAGCGGTGGAGCATGTGGTTTAATTCGAA |
| | GCAACGCGAAGAACCTTACCAAGTCTTGACATCCTCTTGACCGGC |
| | GTGTAACGGCGCCTTCCCTTCGGGGCAAGAGAGACAGGTGGTGCA |
| | TGGTTGTCGTCAGCTCGTGTCGTGAGATGTTGGGTTAAGTCCCGC |
| | AACGAGCGCAACCCTTATCCTTAGTAGCCAGCAGGTAAAGCTGGG |
| | CACTCTAGGGAGACTGCCAGGGATAACCTGGAGGAAGGTGGGGAT |
| | GACGTCAAATCATCATGCCCCTTATGATTTGGGCTACACACGTGC |
| | TACAATGGCGTAAACAAAGGGAAGCAAGACAGTGATGTGGAGCAA |
| | ATCCCAAAAATAACGTCCCAGTTCGGACTGTAGTCTGCAACCCGA |
| | CTACACGAAGCTGGAATCGCTAGTAATCGCGAATCAGAATGTCGC |
| | GGTGAATACGTTCCCGGGTCTTGTACACACCGCCCGTCACACCAT |
| | GGGAGTCAGCAACGCCCGAAGTCAGTGACCCAACTCGCAAGAGAG |
| | GGAGCTGCCGAAGGCGGGGCAGGTAACTGGGGTGAAGTCGTAACA |
| | AGGTAGCCGTATCGGAAGGTGCGGCTGGATCACCTCCTTTCTAAG |
| | GAAGAAGAAGTAGGGGTTTATGTATTATTGAGTCCTTGATTAAAA |
| | GGTGCTTAAAATTTTCCGGTGGCGATGCGCTTAGGGG |
| 6 | AGAGTTTGATCCTGGCTCAGGATGAACGCTGGCGGCATGCCTAAT |
| | ACATGCAAGTCGAACGAAGTGAAGATAGCTTGCTATCGGAGCTTA |
| | GTGGCGAACGGGTGAGTAACACGTAGATAACCTGCCTGTATGACC |
| | GGGATAACAGTTGGAAACGACTGCTAATACCGGATAGGCAGAGAG |
| | GAGGCATCTCTTCTCTGTTAAAGTTGGGATACAACGCAAACAGAT |
| | GGATCTGCGGTGCATTAGCTAGTTGGTGAGGTAACGGCCCACCAA |
| | GGCGATGATGCATAGCCGGCCTGAGAGGGCGAACGGCCACATTGG |
| | GACTGAGACACGGCCCAAACTCCTACGGGAGGCAGCAGTAGGGAA |
| | TTTTCGGCAATGGGGGAAACCCTGACCGAGCAATGCCGCGTGAGT |
| | GAAGACGGCCTTCGGGTTGTAAAGCTCTGTTGTAAGGGAAGAACG |
| | GCATAGAGAGGGAATGCTCTATGAGTGACGGTACCTTACCAGAAA |
| | GCCACGGCTAACTACGTGCCAGCAGCCGCGGTAATACGTAGGTGG |
| | CAAGCGTTATCCGGAATTATTGGGCGTAAAGGGTGCGTAGGCGGC |
| | TGGATAAGTCTGAGGTAAAAGCCCGTGGCTCAACCACGGTAAGCC |
| | TTGGAAACTGTCTGGCTGGAGTGCAGGAGAGGACAATGGAATTCC |
| | ATGTGTAGCGGTAAAATGCGTAGATATATGGAGGAACACCAGTGG |
| | CGAAGGCGGTTGTCTGGCCTGTAACTGACGCTGAAGCACGAAAGC |
| | GTGGGGAGCAAATAGGATTAGATACCCTAGTAGTCCACGCCGTAA |
| | ACGATGAGAACTAAGTGTTGGGGAAACTCAGTGCTGCAGTTAACG |
| | CAATAAGTTCTCCGCCTGGGGAGTATGCACGCAAGTGTGAAACTC |
| | AAAGGAATTGACGGGGGCCCGCACAAGCGGTGGAGTATGTGGTTT |
| | AATTCGACGCAACGCGAAGAACCTTACCAGGCCTTGACATGGTAT |
| | CAAAGGCCCTAGAGATAGGGAGATAGTTATGATACACACAGGTGG |
| | TGCATGGTTGTCGTCAGCTCGTGTCGTGAGATGTTGGGTTAAGTC |
| | CCGCAACGAGCGCAACCCTTGTTTCTAGTTACCAACAGTAAGATG |
| | GGGACTCTAGAGAGACTGCCGGTGACAAACCGGAGGAAGGTGGGG |
| | ATGACGTCAAATCATCATGCCCCTTATGGCCTGGGCTACACACGT |
| | ACTACAATGGCGTCTACAAAGAGCAGCGAGCAGGTGACTGTAAGC |
| | GAATCTCATAAAGGACGTCTCAGTTCGGATTGAAGTCTGCAACTC |
| | GACTTCATGAAGTCGGAATCGCTAGTAATCGCGGATCAGCATGCC |
| | GCGGTGAATACGTTCTCGGGCCTTGTACACACCGCCCGTCAAACC |
| | ATGGGAGTTGATAATACCCGAAGCCGGTGGCCTAACCGAAAGGAG |
| | GGAGCCGTCGAAGGTAGGATCGATGACTGGGGTTAAGTCGTAACA |
| | AGGTATCCC |
| 7 | AAAAAAGATACACGAACGTTCTTTATAAAAGGACACAGTAAAACA |
| | AGGATAAAAAAGCTAGCGCTTTTTAGATCCAAGATTGAACATGAG |
| | AGTTTGATCCTGGCTCAGGATGAACGCTGGCGGCGTGCTTAACAC |
| | ATGCAAGTCGAACGAAGCATTTTGGAAGGAAGTTTTCGGATGGAA |
| | TTCCTTAATGACTGAGTGGCGGACGGGTGAGTAACGCGTGGGGAA |
| | CCTGCCCTATACAGGGGGATAACAGCTGGAAACGGCTGCTAATAC |
| | CGCATAAGCGCACAGAATCGCATGATTCGGTGTGAAAAGCTCCGG |
| | CAGTATAGGATGGTCCCGCGTCTGATTAGCTGGTTGGCGGGGTAA |
| | CGGCCCACCAAGGCGACGATCAGTAGCCGGCTTGAGAGAGTGGAC |
| | GGCCACATTGGGACTGAGACACGGCCCAAACTCCTACGGGAGGCA |
| | GCAGTGGGGAATATTGCACAATGGGGGAAACCCTGATGCAGCGAC |
| | GCCGCGTGAGTGAAGAAGTATTTCGGTATGTAAAGCTCTATCAGC |
| | AGGGAAGAAAAAAGACGGTACCTGACTAAGAAGCCCCGGCTAACT |
| | ACGTGCCAGCAGCCGCGGTAATACGTAGGGGGCAAGCGTTATCCG |
| | GAATTACTGGGTGTAAAGGGTGCGTAGGTGGCATGGTAAGTCAGA |
| | AGTGAAAGCCCGGGGCTTAACCCCGGGACTGCTTTTGAAACTGTC |
| | ATGCTGGAGTGCAGGAGAGGTAAGCGGAATTCCTAGTGTAGCGGT |
| | GAAATGCGTAGATATTAGGAGGAACACCAGTGGCGAAGGCGGCTT |
| | ACTGGACTGTCACTGACACTGATGCACGAAAGCGTGGGGAGCAAA |
| | CAGGATTAGATACCCTGGTAGTCCACGCCGTAAACGATGAATACT |
| | AGGTGTCGGGGCCGTAGAGGCTTCGGTGCCGCAGCAAACGCAGTA |
| | AGTATTCCACCTGGGGAGTACGTTCGCAAGAATGAAACTCAAAGG |
| | AATTGACGGGGACCCGCACAAGCGGTGGAGCATGTGGTTTAATTC |
| | GAAGCAACGCGAAGAACCTTACCTGGTCTTGACATCTAACTGACC |
| | GGTTCGTAATGGGACCTTTCCTTCGGGACAGTTAAGACAGGTGGT |
| | GCATGGTTGTCGTCAGCTCGTGTCGTGAGATGTTGGGTTAAGTCC |
| | CGCAACGAGCGCAACCCCTATCTTTAGTAGCCAGCATATAAGGTG |
| | GGCACTCTAGAGAGACTGCCAGGGATAACCTGGAGGAAGGTGGGG |
| | ACGACGTCAAATCATCATGCCCCTTATGGCCAGGGCTACACACGT |
| | GCTACAATGGCGTAAACAAAGGGAAGCGAAGTCGTGAGGCGAAGC |
| | AAATCCCAGAAATAACGTCTCAGTTCGGATTGTAGTCTGCAACTC |
| | GACTACATGAAGCTGGAATCGCTAGTAATCGTGAATCAGAATGTC |
| | ACGGTGAATACGTTCCCGGGTCTTGTACACACCGCCCGTCACACC |
| | ATGGGAGTCAGTAACGCCCGAAGTCAGTGACCCAACCTTATAGGA |
| | GGGAGCTGCCGAAGGTGGGACCGATAACTGGGGTGAAGTCGTAAC |
| | AAGGTAGCCGTATCGGAAGGTGCGGCTGGATCACCTCCTTTCTAA |
| | GGAAGAAGAAGTAGGGGTTGTCTTATTGTTTAGTTATCAATGATA |
| | ACAAAAAGTATCAAGCAAGGAAGTTCTATTGCACTAAA |
| 8 | ATCAAAGAGTTTGATCCTGGCTCAGGACGAACGCTGGCGGCGCGC |
| | CTAACACATGCAAGTCGAACGGAGCTTACGTTTTGAAGTTTTCGG |
| | ATGGAtGAATGTAAGCTTAGTGGCGGACGGGTGAGTAACACGTGA |
| | GCAACCTGCCTTTCAGAGGGGGATAACAGCCGGAAACGGCTGCTA |
| | ATACCGCATGATGTTGCGGGGGCACATGCCCCTGCAACCAAAGGA |
| | GCAATCCGCTGAAAGATGGGCTCGCGTCCGATTAGCCAGTTGGCG |
| | GGGTAACGGCCCACCAAAGCGACGATCGGTAGCCGGACTGAGAGG |
| | TTGAACGGCCACATTGGGACTGAGACACGGCCCAGACTCCTACGG |
| | GAGGCAGCAGTgGGGGATATTGCACAATGGGCGAAAGCCTGATGC |
| | AGCGACGCCGCGTGAGGGAAGACGGTCTTCGGATTGTAAACCTCT |
| | GTCTTTGGGGAAGAAAATGACGGTACCCAAAGAGGAAGCTCCGGC |
| | TAACTACGTGCCAGCAGCCGCGGTAATACGTAGGGAGCAAGCGTT |
| | GTCCGGAATTACTGGGTGTAAAGGGAGCGTAGGCGGGATGGCAAG |
| | TAGAATGTTAAATCCATCGGCTCAACCGGTGGCTGCGTTCTaAAC |
| | TGCCGTTCTTGAGTGAAGTAGAGGCAGGCGGAATTCCTAGTGTAG |
| | CGGTGAAATGCGTAGATATTAGGAGGAACACCAGTGGCGAAGGCG |
| | GCCTGCTGGGCTTTAACTGACGCTGAGGCTCGAAAGCGTGGGGAG |
| | CAAACAGGATTAGATACCCTGGTAGTCCACGCCGTAAACGATGAT |
| | TACTAGGTGTGGGGGGACTGACCCCTTCCGTGCCGCAGTTAACAC |
| | AATAAGTAATCCACCTGGGGAGTACGGCCGCAAGGTTGAAACTCA |
| | AAGGAATTGACGGGGGCCCGCACAAGCAGTGGAGTATGTGGTTTA |
| | ATTCGAAGCAACGCGAAGAACCTTACCAGGTCTTGACATCGGATG |
| | CATAGCCTAGAGATAGGTGAAGccCTTCGGGGCATCCAGACAGGT |
| | GGTGCATGGTTGTCGTCAGCTCGTGTCGTGAGATGTTGGGTTAAG |
| | TCCCGCAACGAGCGCAACCCTTATTATTAGTTGCTACGCAAGAGC |
| | ACTCTAATGAGACTGCCGTTGACaAAACGGAGGAAGGTGGGGATG |
| | ACGTCAAATCATCATGCCCCTTATGACCTGGGCTACACACGTACT |
| | ACAATGGCACTAAAACAGAGGGCGGCGACACCGCGAGGTGAAGCG |
| | AATCCCGAAAAAGTGTCTCAGTTCAGATTGCAGGCTGCAACCCGC |
| | CTGCATGAAGTCGGAATTGCTAGTAATCGCGGATCAGCATGCCGC |
| | GGTGAATACGTTCCCGGGCCTTGTACACACCGCCCGTCACACCAT |
| | gGGAGTCGGTAACACCCGAAGCCAGTAGCCTAACCGCAAGGGGGG |
| | CGCTGTCGAAGGTGGGATTGATGACTGGGGTGAAGTCGTAACAAG |
| | GTAGCCGTATCGGAAGGTGCGGCTGGATCACCTCCTTTCTAAGGA |
| | GCGAAGCCGGCGGAGAGCCGGCGGACTCTGGTCAGACAGGG |
| 9 | AACAGGAACTTTAAGTTCCACAAACCAGACAACAGTAACGGGATA |
| | GATTAGCGAGAGTTGATCTTGACCAGAACAAGTTCTTATTACGAG |
| | AGTTTGATCCTGGCTCAGGATGAACGCTGGCGGCGTGCCTAACAC |
| | ATGCAAGTCGAGCGAAGCGCTGTTTTCAGAATCTTCGGAGGAAGA |
| | GGACAGTGACTGAGCGGCGGACGGGTGAGTAACGCGTGGGCAACC |
| | TGCCTCATACAGGGGGATAACAGTTAGAAATGACTGCTAATACCG |
| | CATAAGCGCACAGGACCGCATGGTGTAGTGTGAAAAACTCCGGTG |
| | GTATGAGATGGACCCGCGTCTGATTAGGTAGTTGGTGGGGTAAAG |
| | GCCTACCAAGCCGACGATCAGTAGCCGACCTGAGAGGGTGACCGG |
| | CCACATTGGGACTGAGACACGGCCCAAACTCCTACGGGAGGCAGC |
| | AGTGGGGAATATTGCACAATGGGGGAAACCCTGATGCAGCGACGC |
| | CGCGTGAAGGAAGAAGTATTTCGGTATGTAAACTTCTATCAGCAG |
| | GGAAGAAGATGACGGTACCTGAGTAAGAAGCACCGGCTAAATACG |
| | TGCCAGCAGCCGCGGTAATACGTATGGTGCAAGCGTTATCCGGAT |
| | TTACTGGGTGTAAAGGGAGCGTAGACGGATAGGCAAGTCTGGAGT |
| | GAAAACCCAGGGCTCAACCCTGGGACTGCTTTGGAAACTGCAGAT |
| | CTGGAGTGCCGGAGAGGTAAGCGGAATTCCTAGTGTAGCGGTGAA |
| | ATGCGTAGATATTAGGAGGAACACCAGTGGCGAAGGCGGCTTACT |
| | GGACGGTGACTGACGTTGAGGCTCGAAAGCGTGGGGAGCAAACAG |
| | GATTAGATACCCTGGTAGTCCACGCCGTAAACGATGACTACTAGG |
| | TGTCGGTGTGCAAAGCACATCGGTGCCGCAGCAAACGCAATAAGT |
| | AGTCCACCTGGGGAGTACGTTCGCAAGAATGAAACTCAAAGGAAT |
| | TGACGGGGACCCGCACAAGCGGTGGAGCATGTGGTTTAATTCGAA |
| | GCAACGCGAAGAACCTTACCTGGTCTTGACATCCGGATGACGGGC |
| | GAGTAATGTCGCCGTCCCTTCGGGGCATCCGAGACAGGTGGTGCA |
| | TGGTTGTCGTCAGCTCGTGTCGTGAGATGTTGGGTTAAGTCCCGC |
| | AACGAGCGCAACCCTTATCTTCAGTAGCCAGCATATAAGGTGGGC |
| | ACTCTGGAGAGACTGCCAGGGAGAACCTGGAGGAAGGTGGGGATG |
| | ACGTCAAATCATCATGCCCCTTATGGCCAGGGCTACACACGTGCT |
| | ACAATGGCGTAAACAAAGGGAAGCGAGAGGGTGACCTGGAGCGAA |
| | TCCCAAAAATAACGTCTCAGTTCGGATTGTAGTCTGCAACTCGAC |
| | TACATGAAGCTGGAATCGCTAGTAATCGCGGATCAGCATGCCGCG |
| | GTGAATACGTTCCCGGGTCTTGTACACACCGCCCGTCACACCATG |
| | GGAGTCAGTAACGCCCGAAGCCAGTGACCCAACCTTAGAGGAGGG |
| | AGCTGTCGAAGGCGGGACGGATAACTGGGGTGAAGTCGTAACAAG |
| | GTAGCCGTATCGGAAGGTGCGGCTGGATCACCTCCTTTCTAAGGG |
| | AAGAAGTAAGGGACGTTGTCTATTGTTGAGCGATCGAAGAGGGGA |
| | GACCCGAAGAGGAGCTCAGAAGCAGTTGGTGGTGA |
| 10 | GAACCTTGAAAATCAAAGATTGAACAGTATGTAAAACCCTGAAAA |
| | TTCTAAAAAATAAAGGTCTGGTTTAGACCTTTGGATTTGAGAAAA |
| | TTCAGAACAAAACCAAGTAATGAAGGTTAAAGATAACTAGCCAAG |
| | CTAAGTAGTCTAAGACCAAGAATCAACTTTTAATTTGAGAGTTTG |
| | ATCCTGGCTCAGGATGAACGCTGGCGGCGTGCCTAACACATGCAA |
| | GTCGAACGAAGCATTTTAGATGAAGTTTTCGGATGGATTCTGAGA |
| | TGACTGAGTGGCGGACGGGTGAGTAACACGTGGATAACCTGCCTC |
| | ACACTGGGGGACAACAGTTAGAAATGACTGCTAATACCGCATAAG |
| | CGCACAGTaCCGCATGGTACGGTGTGAAAAACTCCGGTGGTGTGA |
| | GATGGATCCGCGTCTGATTAGCCAGTTGGCGGGGTAACGGCCCAC |
| | CAAAGCGACGATCAGTAGCCGACCTGAGAGGGTGACCGGCCACAT |
| | TGGGACTGAGACACGGCCCAAACTCCTACGGGAGGCAGCAGTGGG |
| | GAATATTGCACAATGGGCGAAAGCCTGATGCAGCGACGCCGCGTG |
| | AGTGAAGAAGTATTTCGGTATGTAAAGCTCTATCAGCAGGGAAGA |
| | TAATGACGGTACCTGACTAAGAAGCCCCGGCTAACTACGTGCCAG |
| | CAGCCGCGGTAATACGTAGGGGGCAAGCGTTATCCGGATTTACTG |
| | GGTGTAAAGGGAGCGTAGACGGCATGGCAAGTCTGAAGTGAAAAC |
| | CCAGGGCTCAACCCTGGGACTGCTTTGGAAACTGTCAAGCTAGAG |
| | TGCAGGAGAGGTAAGTGGAATTCCTAGTGTAGCGGTGAAATGCGT |
| | AGATATTAGGAGGAACACCAGTGGCGAAGGCGGCTTACTGGACTG |
| | TAACTGACGTTGAGGCTCGAAAGCGTGGGGAGCAAACAGGATTAG |
| | ATACCCTGGTAGTCCACGCCGTAAACGATGAGTGCTAGGTGTTGG |
| | GGGGCAAAGCCCTTCGGTGCCGTCGCAAACGCAATAAGCACTCCA |
| | CCTGGGGAGTACGTTCGCAAGAATGAAACTCAAAGGAATTGACGG |
| | GGACCCGCACAAGCGGTGGAGCATGTGGTTTAATTCGAAGCAACG |
| | CGAAGAACCTTACCAAGTCTTGACATCCTCTTGACCGGCGTGTAA |
| | CGGCGCCTTTCCTTCGGGACAAGAGAGACAGGTGGTGCATGGTTG |
| | TCGTCAGCTCGTGTCGTGAGATGTTGGGTTAAGTCCCGCAACGAG |
| | CGCAACCCTTATCCTTAGTAGCCAGCATTAAGATGGGCACTCTAG |
| | GGAGACTGCCAGGGACAACCTGGAGGAAGGTGGGGATGACGTCAA |
| | ATCATCATGCCCCTTATGATTTGGGCTACACACGTGCTACAATGG |
| | CGTAAACAAAGGGAAGCGACCCTGCGAAGGTGAGCAAATCTCAAA |
| | AATAACGTCCCAGTTCGGACTGTAGTCTGCAACCCGACTACACGA |
| | AGCTGGAATCGCTAGTAATCGCGAATCAGAATGTCGCGGTGAATA |
| | CGTTCCCGGGTCTTGTACACACCGCCCGTCACACCATGGGAGTCA |
| | GCAACGCCCGAAGTCAGTGACCCAACCGAAAGGAGGGAGCTGCCG |
| | AAGGCGGGGCAGGTAACTGGGGTGAAGTCGTAACAAGGTAGCCGT |
| | ATCGGAAGGTGCGGCTGGATCACCTCCTTTCTAAGGAAGAAGAAG |
| | TAAGGGTTTTATATACTGTTGAATCTTGGATTTGCCAAGGT |
| 11 | CCGgATCAATATGTAGGTTTAAAAaCAAAGGAAATCGCAGATATA |
| | GTGCACGATCGgATTCAAAACAAGATTGATGAAAATATATAAAAA |
| | TATCTGGAAAAACAGGCTTGACAACCCAATTCTtATTTAGTATAA |
| | TTGACCATGCGCTTGACATGAGCATGTCCAAGCCGCATAGTTCAA |
| | CTTAATAGTTTTAAGTAATAACTGCGGAGAAATACTCAAGAGGCC |
| | GAAGAGGCGCCCCTGCTAAGGGTGTAGGTCGGGCAACCGGCGCGA |
| | GGGTTCAAATCCCTCTTTCTCCGTTTTCTTTTTtGAAAAAAAaCC |
| | TATTGAATATATTGTTTGAAAAACTTCAAAAAaGGTGTTGACACC |
| | GAATGAAAGTTATGATATGATATTCAAGCTGCTGAAAACGAGCGG |
| | CGGTTTCAAAAAAaGAAATTAAAAAAaTAAAAAAaGTTGTTGACA |
| | AACAAAAACAATTATGGTAAGATATAAAAGTTGCTGCTGAGAACA |
| | AACGACAGCGTTCAACAACAAAGAACCTTGAAAACTGAACAGTAT |
| | GTAAAACCTTGAAAATTCTTAAAGAACAAAGGCTTTTGGTTAAGC |
| | CTAAAAGTTCGAGAGAACATTCAGAACAAAAAAaCCAAACAACAG |
| | TAATACGGTTTAAAAATTAGCCAAGCGAATTTTTGAACGGAATCA |
| | AAACTTAAACATGAGAGTTTGATCCTAGCTCAGGATGAACGCTGG |
| | CGGCGTGCCTAACACATGCAAGTCGAACGAAGCAATTTAACGGAA |
| | GTTTTCGGATGGAAGTTGAATTGACTGAGTGGCGGACGGGTGAGT |
| | AACGCGTGGGTAACCTGCCTTGTACTGGGGGACAACAGTTAGAAA |
| | TGACTGCTAATACCGCATAAGCGCACAGTATCGCATGATACAGTG |
| | TGAAAAACTCCGGTGGTACAAGATGGACCCGCGTCTGATTAGCTA |
| | GTTGGTAAGGTAACGGCTTACCAAGGCGACGATCAGTAGCCGACC |
| | TGAGAGGGTGACCGGCCACATTGGGACTGAGACACGGCCCAAACT |
| | CCTACGGGAGGCAGCAGTGGGGAATATTGCACAATGGGCGAAAGC |
| | CTGATGCAGCGACGCCGCGTGAGTGAAGAAGTATTTCGGTATGTA |
| | AAGCTCTATCAGCAGGGAAGAAAATGACGGTACCTGACTAAGAAG |
| | CCCCGGCTAACTACGTGCCAGCAGCCGCGGTAATACGTAGGGGGC |
| | AAGCGTTATCCGGATTTACTGGGTGTAAAGGGAGCGTAGACGGTA |
| | AAGCAAGTCTGAAGTGAAAGCCCGCGGCTCAACTGCGGGACTGCT |
| | TTGGAAACTGTTTAACTGGAGTGTCGGAGAGGTAAGTGGAATTCC |
| | TAGTGTAGCGGTGAAATGCGTAGATATTAGGAGGAACACCAGTGG |
| | CGAAGGCGACTTACTGGACGATAACTGACGTTGAGGCTCGAAAGC |
| | GTGGGGAGCAAACAGGATTAGATACCCTGGTAGTCCACGCCGTAA |
| | ACGATGAATACTAGGTGTTGGGGAGCAAAGCTCTTCGGTGCCGTC |
| | GCAAACGCAGTAAGTATTCCACCTGGGGAGTACGTTCGCAAGAAT |
| | GAAACTCAAAGGAATTGACGGGGACCCGCACAAGCGGTGGAGCAT |
| | GTGGTTTAATTCGAAGCAACGCGAAGAACCTTACCAGGTCTTGAC |
| | ATCGATCCGACGGGGGAGTAACGTCCCCTTCCCTTCGGGGCGGAG |
| | AAGACAGGTGGTGCATGGTTGTCGTCAGCTCGTGTCGTGAGATGT |
| | TGGGTTAAGTCCCGCAACGAGCGCAACCCTTATTCTAAGTAGCCA |
| | GCGGTTCGGCCGGGAACTCTTGGGAGACTGCCAGGGATAACCTGG |
| | AGGAAGGTGGGGATGACGTCAAATCATCATGCCCCTTATGATCTG |
| | GGCTACACACGTGCTACAATGGCGTAAACAAAGAGAAGCAAGACC |
| | GCGAGGTGGAGCAAATCTCAAAAATAACGTCTCAGTTCGGACTGC |
| | AGGCTGCAACTCGCCTGCACGAAGCTGGAATCGCTAGTAATCGCG |
| | AATCAGAATGTCGCGGTGAATACGTTCCCGGGTCTTGTACACACC |
| | GCCCGTCACACCATGGGAGTCAGTAACGCCCGAAGTCAGTGACCC |
| | AACCGCAAGGAGGGAGCTGCCGAAGGCGGGACCGATAACTGGGGT |
| | GAAGTCGTAACAAGGTAGCCGTATCGGAAGGTGCGGCTGGATCAC |
| | CTCCTTTCTAAGGAAGAAGAAGTAGAGGTTTTATATACTGTTGAG |
| | TTTTCAAGCTCAGACACTTCCGGTGTCGATGCGCTCAGGGGTCAC |
| | ACCCGTTCCCATCCCGAACACGATGGTTAAGACCTGAGCGGCCGA |
| | TGATACTATATTGGAGACGATATGGGAAAGCAGGTGGATGCCGGA |
| | TTTTTCTTTAAAAAACTCACTCGTAAGAGTGTTTTATATAGAACG |
| | GACACATCTGAATGGATGAATTCGTAGTTTCTTTAGATGAAAAAG |
| | ACATCTGTGACGACGATGTGTTAACCCACTCCAGCAGGGAAGTGC |
| | TGTTCCATATAACTGGTTTTTACCAGTGATTAGTGAATTTGAGTT |
| | TATCGAGTTTGCTAATGACTGATAAAGTTCAGTCAGATATGTACC |
| | TTGAAAACTGCATATTGAAATAATATCTAGATAGAGTTTCGAAAC |
| | TCTTCCGAGTGGCTTGCCACAAGGGAGAAAAGAGAGACAAAATCA |
| | AGACATCCGAGGTGTTACACGAAAGTGTAACCAAAACAAACCAAA |
| | AATTTTATTGTAAACCTAAACCAAAGATTCAACGCTATGAATCTT |
| | AGACT |
| 12 | ATTGAAAACTAAACAGAATTAAGAAACACACGTCAATATCCAGAA |
| | GGATAAAAAAGAAAAAAGAGCTAAACAAACGAAGAGTTGTTTAAA |
| | TAAACAATGGAGAGTTTGATCCTGGCTCAGGATGAACGCTGGCGG |
| | CGTGCCTAATACATGCAAGTCGAACGCGAGCACTTGTGCTCGAGT |
| | GGCGAACGGGTGAGTAATACATAAGTAACCTGCCCTAGACAGGGG |
| | GATAACTATTGGAAACGATAGCTAAGACCGCATAGGTACGGACAC |
| | TGCATGGTGACCGTATTAAAAGTGCCTCAAAGCACTGGTAGAGGA |
| | TGGACTTATGGCGCATTAGCTGGTTGGCGGGGTAACGGCCCACCA |
| | AGGCGACGATGCGTAGCCGACCTGAGAGGGTGACCGGCCACACTG |
| | GGACTGAGACACGGCCCAGACTCCTACGGGAGGCAGCAGTAGGGA |
| | ATTTTCGGCAATGGGGGAAACCCTGACCGAGCAACGCCGCGTGAA |
| | GGAAGAAGGTTTTCGGATTGTAAACTTCTGTTATAAAGGAAGAAC |
| | GGCGGCTACAGGAAATGGTAGCCGAGTGACGGTACTTTATTAGAA |
| | AGCCACGGCTAACTACGTGCCAGCAGCCGCGGTAATACGTAGGTG |
| | GCAAGCGTTATCCGGAATTATTGGGCGTAAAGAGGGAGCAGGCGG |
| | CAGCAAGGGTCTGTGGTGAAAGCCTGAAGCTTAACTTCAGTAAGC |
| | CATAGAAACCAGGCAGCTAGAGTGCAGGAGAGGATCGTGGAATTC |
| | CATGTGTAGCGGTGAAATGCGTAGATATATGGAGGAACACCAGTG |
| | GCGAAGGCGACGATCTGGCCTGCAACTGACGCTCAGTCCCGAAAG |
| | CGTGGGGAGCAAATAGGATTAGATACCCTAGTAGTCCACGCCGTA |
| | AACGATGAGTACTAAGTGTTGGATGTCAAAGTTCAGTGCTGCAGT |
| | TAACGCAATAAGTACTCCGCCTGAGTAGTACGTTCGCAAGAATGA |
| | AACTCAAAGGAATTGACGGGGGCCCGCACAAGCGGTGGAGCATGT |
| | GGTTTAATTCGAAGCAACGCGAAGAACCTTACCAGGTCTTGACAT |
| | ACTCATAAAGGCTCCAGAGATGGAGAGATAGCTATATGAGATACA |
| | GGTGGTGCATGGTTGTCGTCAGCTCGTGTCGTGAGATGTTGGGTT |
| | AAGTCCCGCAACGAGCGCAACCCTTATCGTTAGTTACCATCATTA |
| | AGTTGGGGACTCTAGCGAGACTGCCAGTGACAAGCTGGAGGAAGG |
| | CGGGGATGACGTCAAATCATCATGCCCCTTATGACCTGGGCTACA |
| | CACGTGCTACAATGGATGGTGCAGAGGGAAGCGAAGCCGCGAGGT |
| | GAAGCAAAACCCATAAAACCATTCTCAGTTCGGATTGTAGTCTGC |
| | AACTCGACTACATGAAGTTGGAATCGCTAGTAATCGCGAATCAGC |
| | ATGTCGCGGTGAATACGTTCTCGGGCCTTGTACACACCGCCCGTC |
| | ACACCACGAGAGTTGATAACACCCGAAGCCGGTGGCCTAACCGCA |
| | AGGAAGGAGCTGTCTAAGGTGGGATTGATGATTGGGGTGAAGTCG |
| | TAACAAGGTATcCCTACGGGAACGTGGGGATGGATCACCTCCTTT |
| | CTAAGGAGAAACGCATGATCGAAGATGATGCACAAGAGAAGTGAT |
| | GTTTCGAAGATACTGTTTAGTTTTGAGTGATACTCAGGT |
| 13 | ACTGAGATTAATCTCGGTGGTAAGAACAGTAAAAAGGGATAGAAT |
| | TAGCTAGTAGTTAATTCTGACCCGGATACAAACACTTTTAACGAG |
| | AGTTTGATCCTGGCTCAGGATGAACGCTGGCGGCGTGCTTAACAC |
| | ATGCAAGTCGAGCGAAGCGCTTTACTTAGATTTCTTCGGATTGAA |
| | AAGTTTTGCGACTGAGCGGCGGACGGGTGAGTAACGCGTGGGTAA |
| | CCTGCCTCATACAGGGGGATAACAGTTAGAAATGACTGCTAATAC |
| | CGCATAAGACCACAGTACCGCATGGTACAGTGGGAAAAACTCCGG |
| | TGGTATGAGATGGACCCGCGTCTGATTAGCTAGTTGGTAAGGTAA |
| | CGGCTTACCAAGGCGACGATCAGTAGCCGACCTGAGAGGGTGACC |
| | GGCCACATTGGGACTGAGACACGGCCCAAACTCCTACGGGAGGCA |
| | GCAGTGGGGAATATTGCACAATGGGGGAAACCCTGATGCAGCGAC |
| | GCCGCGTGAAGGATGAAGTATTTCGGTATGTAAACTTCTATCAGC |
| | AGGGAAGAAAATGACGGTACCTGACTAAGAAGCCCCGGCTAACTA |
| | CGTGCCAGCAGCCGCGGTAATACGTAGGGGGCAAGCGTTATCCGG |
| | ATTTACTGGGTGTAAAGGGAGCGTAGACGGTTATGTAAGTCTGAT |
| | GTGAAAACCCGGGGCTCAACCCCGGGACTGCATTGGAAACTATGT |
| | AACTAGAGTGTCGGAGAGGTAAGTGGAATTCCTAGTGTAGCGGTG |
| | AAATGCGTAGATATTAGGAGGAACACCAGTGGCGAAGGCGGCTTA |
| | CTGGACGATGACTGACGTTGAGGCTCGAAAGCGTGGGGAGCAAAC |
| | AGGATTAGATACCCTGGTAGTCCACGCCGTAAACGATGCATACTA |
| | GGTGTCGGGTGGCAAAGCCATTCGGTGCCGCAGCAAACGCAATAA |
| | GTATGCCACCTGGGGAGTACGTTCGCAAGAATGAAACTCAAAGGA |
| | ATTGACGGGGACCCGCACAAGCGGTGGAGCATGTGGTTTAATTCG |
| | AAGCAACGCGAAGAACCTTACCTGCTCTTGACATCCCCCTGACCG |
| | GCGCGTAATGGTGCCTTTCCTTCGGGACAGGGGAGACAGGTGGTG |
| | CATGGTTGTCGTCAGCTCGTGTCGTGAGATGTTGGGTTAAGTCCC |
| | GCAACGAGCGCAACCCTTATCTTTAGTAGCCAGCGGTTTGGCCGG |
| | GCACTCTAGAGAGACTGCCAGGGATAACCTGGAGGAAGGTGGGGA |
| | TGACGTCAAATCATCATGCCCCTTATGAGCAGGGCTACACACGTG |
| | CTACAATGGCGTAAACAAAGGGAGGCGAAGCCGCGAGGTGGAGCA |
| | AATCCCAAAAATAACGTCTCAGTTCGGATTGTAGTCTGCAACTCG |
| | ACTACATGAAGCTGGAATCGCTAGTAATCGCGAATCAGAATGTCG |
| | Cggtgaatacgttcccgggtcttgtacacaccgcccgtcacacca |
| | tgggagttggtaacgcccgaagtcagtgacccaaccGCAAGGAGG |
| | GAGCTGCCGAAGGTGGGACCGATAACTGGGGTGAAGTCGTAACAA |
| | GGTAGCCGTATCGGAAGGTGCGGCTGGATCACCTCCTTTCTAAGG |
| | AAGAAAGAAGTAAAGAGTATGTTGT |
| 14 | TCAACCACAGAACCTTGATAATTGAACAATAGACAACAACCCTGA |
| | AAATTTCTTGTAAAGAGAGAATTTCAGAACAGAACGGGACATCGA |
| | GAGATGTCCGACCTTAAAAACAGTAATAACGGGAAAGAATTAGCC |
| | AAGAGTTGATTCTGACCGCGATCACAACTTTTAACGAGAGTTTGA |
| | TCCTGGCTCAGGATGAACGCTGGCGGCGTGCCTAACACATGCAAG |
| | TCGAACGAAGCGCttccgctagattttcttcggagatgaaggcgg |
| | cTGCGACTGAGTGGCGGACGGGTGAGTAACGCGTGGGCAACCTGC |
| | CTTGCACTGGGGGATAACAGCCAGAAATGGCTGCTAATACCGCAT |
| | AAGACCGAAGCGCCGCATGGCGCAGCGGCCAAAGCCCCGGCGGTG |
| | CAAGATGGGCCCGCGTCTGATTAGGTAGTTGGCGGGGTAACGGCC |
| | CACCAAGCCGACGATCAGTAGCCGACCTGAGAGGGTGACCGGCCA |
| | CATTGGGACTGAGACACGGCCCAGACTCCTACGGGAGGCAGCAGT |
| | GGGGAATATTGCACAATGGGGGAAACCCTGATGCAGCGACGCCGC |
| | GTGAAGGATGAAGTATTTCGGTATGTAAACTTCTATCAGCAGGGA |
| | AGAAGATGACGGTACCTGACTAAGAAGCCCCGGCTAACTACGTGC |
| | CAGCAGCCGCGGTAATACGTAGGGGGCAAGCGTTATCCGGATTTA |
| | CTGGGTGTAAAGGGAGCGTAGACGGCGATGCAAGCCAGATGTGAA |
| | AGCCCGGGGCTCAACCCCGGGACTGCATTTGGAACTGCGTGGCTG |
| | GAGTGTCGGAGAGGCAGGCGGAATTCCTAGTGTAGCGGTGAAATG |
| | CGTAGATATTAGGAGGAACACCAGTGGCGAAGGCGGCCTGCTGGA |
| | CGATGACTGACGTTGAGGCTCGAAAGCGTGGGGAGCAAACAGGAT |
| | TAGATACCCTGGTAGTCCACGCCGTAAACGATGACTACTAGGTGT |
| | CGGGTGGCAAGGCCATTCGGTGCCGCAGCAAACGCAATAAGTAGT |
| | CCACCTGGGGAGTACGTTCGCAAGAATGAAACTCAAAGGAATTGA |
| | CGGGGACCCGCACAAGCGGTGGAGCATGTGGTTTAATTCGAAGCA |
| | ACGCGAAGAACCTTACCTGATCTTGACATCCCGATGCCAAAGCGC |
| | GTAACGCGCTCTTTCTTCGGAACATCGGTGACAGGTGGTGCATGG |
| | TTGTCGTCAGCTCGTGTCGTGAGATGTTGGGTTAAGTCCCGCAAC |
| | GAGCGCAACCCCTATCTTCAGTAGCCAGCATTCCGGATGGGCACT |
| | CTGGAGAGACTGCCAGGGACAACCTGGAGGAAGGTGGGGATGACG |
| | TCAAATCATCATGCCCCTTATGACCAGGGCTACACACGTGCTACA |
| | ATGGCGTAAACAAAGGGAGGCGAACCCGCGAgGGTGGGCAAATCC |
| | CAAAAATAACGTCTCAGTTCGGATTGTAGTCTGCAACTCGACTAC |
| | ATGAAGCTGGAATCGCTAGTAATCGCGAATCAGAATGTCGCGGTG |
| | AATACGTTCCCGGGTCTTGTACACACCGCCCGTCACACCATGGGA |
| | GTCAGTAACGCCCGAAGCCGGTGACCCAACCCGtAtGGGAGGGAG |
| | CCGTCGAAGGTGGGACCGATAACTGGGGTGAAGTCGTAACAAGGT |
| | AGCCGTATCGGAAGGTGCGGCTGGATCACCTCCTTTCTaAGGaag |
| | aGAAGAAGTAAGGGCTGCTGTCTATTGTTCAGTTATCGAGGAATG |
| | CTGGaA |
| 15 | TAGAGTTAATCTTAACTGGGTTAAACACTTAAACAAGAGAGTTTG |
| | ATCCTGGCTCAGGATGAACGCTGGCGGCGTGCCTAACACATGCAA |
| | GTCGAACGGAGTTATGCAGAGGAAGTTTTCGGATGGAATCGGCGT |
| | AACTTAGTGGCGGACGGGTGAGTAACGCGTGGGAAACCTGCCCTG |
| | TACCGGGGGATAACACTTAGAAATAGGTGCTAATACCGCATAAGC |
| | GCACAGCTTCACATGAGGCAGTGTGAAAAACTCCGGTGGTACAGG |
| | ATGGTCCCGCGTCTGATTAGCCAGTTGGCAGGGTAACGGCCTACC |
| | AAAGCGACGATCAGTAGCCGGCCTGAGAGGGTGAACGGCCACATT |
| | GGGACTGAGACACGGCCCAAACTCCTACGGGAGGCAGCAGTGGGG |
| | AATATTGCACAATGGGGGAAACCCTGATGCAGCGACGCCGCGTGA |
| | GTGAAGAAGTATTTCGGTATGTAAAGCTCTATCAGCAGGGAAGAA |
| | AATGACGGTACCTGACTAAGAAGCCCCGGCTAACTACGTGCCAGC |
| | AGCCGCGGTAATACGTAGGGGGCAAGCGTTATCCGGATTTACTGG |
| | GTGTAAAGGGAGCGTAGACGGCATGACAAGCCAGATGTGAAAACC |
| | CAGGGCTCAACCCTGGGACTGCATTTGGAACTGCCAGGCTGGAGT |
| | GCAGGAGAGGTAAGCGGAATTCCTAGTGTAGCGGTGAAATGCGTA |
| | GATATTAGGAGGAACACCAGTGGCGAAGGCGGCTTACTGGACTGT |
| | AACTGACGTTGAGGCTCGAAAGCGTGGGGAGCAAACAGGATTAGA |
| | TACCCTGGTAGTCCACGCGGTAAACGATGATTGCTAGGTGTAGGT |
| | GGGTATGGACCCATCGGTGCCGCAGCTAACGCAATAAGCAATCCA |
| | CCTGGGGAGTACGTTCGCAAGAATGAAACTCAAAGGAATTGACGG |
| | GGACCCGCACAAGCGGTGGAGCATGTGGTTTAATTCGAAGCAACG |
| | CGAAGAACCTTACCAAGTCTTGACATCCCAATGACGTGTCCGTAA |
| | CGGGGCATTCTCTTCGGAGCATTGGAGACAGGTGGTGCATGGTTG |
| | TCGTCAGCTCGTGTCGTGAGATGTTGGGTTAAGTCCCGCAACGAG |
| | CGCAACCCTTATCCTTAGTAGCCAGCAGGTAAAGCTGGGCACTCT |
| | AGGGAGACTGCCGGGGATAACCCGGAGGAAGGCGGGGATGACGTC |
| | AAATCATCATGCCCCTTATGATTTGGGCTACACACGTGCTACAAT |
| | GGCGTAAACAAAGGGAAGCGAGACAGTGATGTTGAGCAAATCCCA |
| | GAAATAACGTCTCAGTTCGGATTGTAGTCTGCAACTCGACTACAT |
| | GAAGCTGGAATCGCTAGTAATCGCGAATCAGCATGTCGCGGTGAA |
| | TACGTTCCcGGGTCTtGTACACACCGCCCGTCACACCATGGGAGT |
| | TGGAAATGCCCGAAGCCTGTGACCTAACCGtAAGGGAGGAGCAGT |
| | CGAAGGCAGGTCTAATAACTGGGGTGAAGTCGTAACAAGGTAGCC |
| | GTATCGGAAGGTGCGGCTGGATCACCTCCTTTCTAAGGCGAAGAA |
| | GTAAGGGTTGTATTACTGTTTAGATGTTAAGgg |
| 16 | AATTTGAGAGTTTGATCCTGGCTCAGGATGAACGCTGGCGGCGTG |
| | CCTAACACATGCAAGTCGAACGAAGCATCttATAGGAAGTTTTCG |
| | GATGGAATATGGGATGACTGAGTGGCGGACGGGTGAGTAACGCGT |
| | GGATAACCTGCCTCACACTGGGGGATAACAGTTAGAAATGGCTGC |
| | TAATACCGCATAAGCGCACAGTACCGCATGGTACGGTGTGAAAAA |
| | CCCAGGTGGTGTGAGATGGATCCGCGTCTGATTAGCCAGTTGGCG |
| | GGGTAACGGCCCACCAAAGCGACGATCAGTAGCCGACCTGAGAGG |
| | GTGACCGGCCACATTGGGACTGAGACACGGCCCAAACTCCTACGG |
| | GAGGCAGCAGTGGGGAATATTGCACAATGGGCGAAAGCCTGATGC |
| | AGCGACGCCGCGTGAGTGAAGAAGTATCTCGGTATGTAAAGCTCT |
| | ATCAGCAGGGAAGAAAATGACGGTACCTGACTAAGAAGCCCCGGC |
| | TAACTACGTGCCAGCAGCCGCGGTAATACGTAGGGGGCAAGCGTT |
| | ATCCGGATTTACTGGGTGTAAAGGGAGCGTAGACGGCGATGCAAG |
| | TCTGAAGTGAAAGCCCGGGGCTCAACCCCGGGACTGCTTTGGAAA |
| | CTGTGTGGCTGGAGTGCAGGAGAGGTAAGTGGAATTCCTAGTGTA |
| | GCGGTGAAATGCGTAGATATTAGGAGGAACACCAGTGGCGAAGGC |
| | GGCTTACTGGACTGTAACTGACGTTGAGGCTCGAAAGCGTGGGGA |
| | GCAAACAGGATTAGATACCCTGGTAGTCCACGCCGTAAACGATGA |
| | ATGCTAGGTGTCGGGGGGCAAAGCCCCTCGGTGCCGCCGCTAACG |
| | CAATAAGCATTCCACCTGGGGAGTACGTTCGCAAGAATGAAACTC |
| | AAAGGAATTGACGGGGACCCGCACAAGCGGTGGAGCATGTGGTTT |
| | AATTCGAAGCAACGCGAAGAACCTTACCAAGTCTTGACATCCCCC |
| | TGACCGGACAGTAACGTGTCCCTTCCTTCGGGACAGGGGAGACAG |
| | GTGGTGCATGGTTGTCGTCAGCTCGTGTCGTGAGATGTTGGGTTA |
| | AGTCCCGCAACGAGCGCAACCCTTATCCTTAGTAGCCAGCAccTC |
| | GGGTGGGCACTCTAGGGAGACTGCCAGGGATAACCTGGAGGAAGG |
| | TGGGGATGACGTCAAATCATCATGCCCCTTATGATTTGGGCTACA |
| | CACGTGCTACAATGGCGTAAACAAAGGGAaGcgGCCCTGCGAAGG |
| | TGAGCAAATCCCAAAAATAACGTCCCAGTTCGGACTGTAGTCTGC |
| | AACCCGACTACACGAAGCTGGAATCGCTAGTAATCGCGGATCAGA |
| | ATGCCGCGGTGAATACGTTCCCGGGTCTTGTACACACCGCCCGTC |
| | ACACCATGGGAGTCAGCAACGCCCGAAGTCAGTGACCCAACCGAA |
| | AGGAGGGAGCTGCCGAAGGCGGGGCAGGTAACTGGGGTGAAGTCG |
| | TAACAAGGTAGCCGTATCGGAAGGTGCGGCTGGATCACCTCCTTT |
| | CTAAGGAAGAAGAAGTAgGGGTTTT |
| 17 | gAaTtGAcAAaCAGTTACGCCTTATGtAtAATAACaTTtgCGcta |
| | CCGAATGGgACgCGCAcTTCaTATTGGTAGAGgATAGTtCAGTTT |
| | tGTGGaGAAAtACtCAaGAGGCTGAAgAGGCGCCCCTGCTAAGGg |
| | tGTAGGtCGTTTATAgcGGCGCGAGGGTTCAAATCCCtcTTtCTC |
| | CGTtCCCTTtACCGATatAAAGGGGAGCTTGaTTAtaGCTTcGCG |
| | GGAAAaGAATcTtAAGaGATTGAGaTtttTTTTTTtCGCACTTTt |
| | GCCGgATGAAAAGAACGAGTAAAaCTTATGGAAAatATAGGTTAA |
| | AAAaGTTGTTGACATTTGACAAAatATGGTGCTATAAtGAAAGTC |
| | CACCGCGAATGGCGATGGAaCAGAGATTTAAAaGAAATAAAAAAA |
| | GTTAAAAAaGTGCTTGACTGGCAGAACAACATGTGATAAGATATC |
| | AACTGTTGCGGCTGAGAAAGCAGCACACAGAACCTTGACAAATAA |
| | ACAGTAATGCAACCcTGAAAATTCTAAAAAaGAAAATTCAGAGAA |
| | CAAGTTTTAAAGCTTCGAGAGAAGACCTTtAAGACACCCAAAAAC |
| | AGTAAGAAATGTTTGGAAACAAACAAACGGTTAAAcATTAGCTAG |
| | AGTtAATCTtAaCCGgATTAAaCACTtAAaCAAGAGAGTTtGATC |
| | CTGGCTCAGGATGAACGCTGGCGGCGTGCCTAACACATGCAAGTC |
| | GAACGAAGTTAgACaGAGGAAGTTTTCGGATGGAATCGGTATAAC |
| | TTAGTGGCGGACGGGTGAGTAACGCGTGGGAAACCTGCCCTGTAC |
| | CGGGGGATAACACTTAGAAATAGGTGCTAATACCGCATAAGCGCA |
| | CGGAACCGCATGGTTCtGTGTGAAAAACTCCGGTGGTACAGGATG |
| | GTCCCGCGTCTGATTAGCCAGTTGGCAGGGTAACGGCCTACCAAA |
| | GCGACGATCAGTAGCCGGCCTGAGAGGGTGAACGGCCACATTGGG |
| | ACTGAGACACGGCCCAAACTCCTACGGGAGGCAGCAGTGGGGAAT |
| | ATTGCACAATGGGGGAAACCCTGATGCAGCGACGCCGCGTGAGTG |
| | AAGAAGTATTTCGGTATGTAAAGCTCTATCAGCAGGGAAGAAAAT |
| | GACGGTACCTGACTAAGAAGCCCCGGCTAACTACGTGCCAGCAGC |
| | CGCGGTAATACGTAGGGGGCAAGCGTTATCCGGATTTACTGGGTG |
| | TAAAGGGAGCGTAGACGGCATGGCAAGCCAGATGTGAAAACCCAG |
| | GGCTCAACCTTGGGATTGCATTTGGAACTGCCAGGCTGGAGTGCA |
| | GGAGAGGTAAGCGGAATTCCTAGTGTAGCGGTGAAATGCGTAGAT |
| | ATTAGGAGGAACACCAGTGGCGAAGGCGGCTTACTGGACTGTAAC |
| | TGACGTTGAGGCTCGAAAGCGTGGGGAGCAAACAGGATTAGATAC |
| | CCTGGTAGTCCACGCGGTAAACGATGATTGCTAGGTGTAGGTGGG |
| | TATGGACCCATCGGTGCCGCAGCTAACGCAATAAGCAATCCACCT |
| | GGGGAGTACGTTCGCAAGAATGAAACTCAAAGGAATTGACGGGGA |
| | CCCGCACAAGCGGTGGAGCATGTGGTTTAATTCGAAGCAACGCGA |
| | AGAACCTTACCAAGTCTTGACATCCCAATGACGCACCTGTAAAGA |
| | GGTGTTCCCTTCGGGGCATTGGAGACAGGTGGTGCATGGTTGTCG |
| | TCAGCTCGTGTCGTGAGATGTTGGGTTAAGTCCCGCAACGAGCGC |
| | AACCCTTATTCTTAGTAGCCAGCAGGTAAAGCTGGGCACTCTAAG |
| | GAGACTGCCGGGGATAACCCGGAGGAAGGCGGGGATGACGTCAAA |
| | TCATCATGCCCCTTATGATTTGGGCTACACACGTGCTACAATGGC |
| | GTAAACAAAGGGAAGCGAGACAGTGATGTGGAGCAAATCCCAGAA |
| | ATAACGTCTCAGTTCGGATTGTAGTCTGCAACTCGACTACATGAA |
| | GCTGGAATCGCTAGTAATCGCGAATCAGCATGTCGCGGTGAATAC |
| | GTTCCCGGGTCTTGTACACACCGCCCGTCACACCATGGGAGTTGG |
| | AAATGCCCGAAGTCTGTGACCTAACCGAAAGGGAGGAGCAGCCGA |
| | AGGCAGGTCTGATAACTGGGGTGAAGTCGTAACAAGGTAGCCGTA |
| | TCGGAAGGTGCGGCTGGATCACCTCCTTTCTAAGGCGAAGAAGTA |
| | GGGGTTGTATTACTGTTTAGATGTT |

1. A medium comprising:
(a) 5-15 g/kg of Proteose Peptone,
(b) 5-15 g/kg of Phytone Peptone,
(c) 15-25 g/kg of Yeast extract,
(d) 9-14 g/kg of M9 salts,
(e) 5-15 g/kg of glucose,
wherein the medium is for culturing a bacterial strain belonging to Clostridium genus.

2. The medium of claim 1, wherein the medium comprises:
(a) 10 g/kg of Proteose Peptone,
(b) 10 g/kg of Phytone Peptone,
(c) 20 g/kg of Yeast extract,
(d) 11.4 g/kg of M9 salts,
(e) 10 g/kg of glucose.

3. The medium of claim 1, wherein the bacterial strain belonging to Clostridium genus is a bacterial strain belonging to Clostridium clusters IV or XIVa.

4. The medium of claim 1, wherein the bacterial strain belonging to Clostridium genus is selected from the group consisting of the strains listed in Table 1.

5. The medium of claim 1, wherein the bacterial strain belonging to Clostridium genus comprises a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

6. The medium of claim 1, wherein the medium is maintained in anaerobic conditions.

7. The medium of claim 1, wherein the medium further comprises at least one bacterium comprising 16S rDNA sequences selected from the group: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

8. A process for culturing bacterial cells, said process comprising:
a) providing a bioreactor,
b) mixing the cells to be cultured with the medium according to claim 1,
c) incubating the resultant mixture.

9. The process of claim 8, wherein the bacterial strain belonging to Clostridium genus is a bacterial strain belonging to Clostridium clusters IV or XIVa.

10. The process of claim 8, wherein the bacterial strain belonging to Clostridium genus is selected from the group consisting of the strains listed in Table 1.

11. The process of claim 8, wherein the bacterial strain belonging to Clostridium genus comprises a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

12. The process of claim 8, wherein the medium is maintained in anaerobic conditions.
